SEQUENCE LISTING (41 sequences)

SEQ ID NO: 1 - protein, 871 aa, Homo sapiens
MKYSCCALVLAVLGTELLGSLCSTVRSPRFRGRIQQERKNIRPNIILVPTDDQDVELGSLQVMNKTRKIMEHGGATFINAFVTTPMCCPSRSSMLTGKYVHNHNVYTNNENCSSPSWQAMHEPRTFAVYLNNTGYRTAFFGKYLNEYNGSYIPPGWREWLGLIKNSRFYNYTVCRNGIKEKHGFDYAKDYFTDLITNESINYFKMSKRMYPHRPVMMVISHAAPHGPEDSAPQFSKLYPNASQHITPSYNYAPNMDKHWIMQYTGPMLPIHMEFTNILQRKRLQTLMSVDDSVERLYNMLVETGELENTYIIYTADHGYHIGQFGLVKGKSMPYDFDIRVPFFIRGPSVEPGSIVPQIVLNIDLAPTILDIAGLDTPPDVDGKSVLKLLDPEKPGNRFRTNKKAKIWRDTFLVERGKFLRKKEESSKNIQQSNHLPKYERVKELCQQARYQTACEQPGQKWQCIEDTSGKLRIHKCKGPSDLLTVRQSTRNLYARGFHDKDKECSCRESGYRASRSQRKSQRQFLRNQGTPKYKPRFVHTRQTRSLSVEFEGEIYDINLEEEEELQVLQPRNIAKRHDEGHKGPRDLQASSGGNRGRMLADSSNAVGPPTTVRVTHKCFILPNDSIHCERELYQSARAWKDHKAYIDKEIEALQDKIKNLREVRGHLKRRKPEECSCSKQSYYNKEKGVKKQEKLKSHLHPFKEAAQEVDSKLQLFKENNRRRKKERKEKRRQRKGEECSLPGLTCFTHDNNHWQTAPFWNLGSFCACTSSNNNTYWCLRTVNETHNFLFCEFATGFLEYFDMNTDPYQLTNTVHTVERGILNQLHVQLMELRSCQGYKQCNPRPKNLDVGNKDGGSYDLHRGQLWDGWEG

SEQ ID NO: 2 - protein, 870 aa, Rattus norvegicus [sequence omitted]

SEQ ID NO: 3 - protein, 867 aa, Coturnix coturnix japonica
MKTSWFALFLAVLSTELLTSHSSTLKSLRFRGRVQQERKNIRPNIILVLTDDQDVELGSLQVMNKTRRIMENGGASFINAFVTTPMCCPSRSSMLTGKYVHNHNIYTNNENCSSPSWQATHEPRTFAVYLNNTGYRTAFFGKYLNEYNGSYIPPGWREWVGLVKNSRFYNYTISRNGNKEKHGFDYAKDYFTDLITNESINYFRMSKRIYPHRPIMMVISHAAPHGPEDSAPQFSELYPNASQHITPSYNYAPNMDKHWIMQYTGPMLPIHMEFTNVLQRKRLQTLMSVDDSMERLYQMLAEMGELENTYIIYTADHGYHIGQFGLVKGKSMPYDFDIRVPFFIRGPSVEPGSVVPQIVLNIDLAPTILDIAGLDTPPDMDGKSVLKLLDLERPGNRFRTNKKTKIWRDTFLVERGKFLRKKEEANKNTQQSNQLPKYERVKELCQQARYQTACEQPGQKWQCTEDASGKLRIHKCKVSSDILAIRKRTRSIHSRGYSGKDKDCNCGDTDFRNSRTQRKNQRQFLRNPSAQKYKPRFVHTRQTRSLSVEFEGEIYDINLEEEELQVLKTRSITKRHNAENDKKAEETDGAPGDTMVADGTDVIGQPSSVRVTHKCFILPNDTIRCERELYQSARAWKDHKAYIDKEIEALQDKIKNLREVRGHLKRRKPDECDCTKQSYYNKEKGVKTQEKIKSHLHPFKEAAQEVDSKLQLFKENRRRKKERKGKKRQKKGDECSLPGLTCFTHDNNHWQTAPFWNLGSFCACTSSNNNTYWCLRTVNDTHNFLFCEFATGFLEFFDMNTDPYQLTNTVHTVERGILNQLHVQLMELRSCQGYKQCNPRPKGLETGNKDGGSYDPHRGQLWDGWEG

SEQ ID NO: 4 - protein, 870 aa, Homo sapiens
MGPPSLVLCLLSATVFSLLGGSSAFLSHHRLKGRFQRDRRNIRPNIILVLTDDQDVELGSMQVMNKTRRIMEQGGAHFINAFVTTPMCCPSRSSILTGKYVHNHNTYTNNENCSSPSWQAQHESRTFAVYLNSTGYRTAFFGKYLNEYNGSYVPPGWKEWVGLLKNSRFYNYTLCRNGVKEKHGSDYSKDYLTDLITNDSVSFFRTSKKMYPHRPVLMVISHAAPHGPEDSAPQYSRLFPNASQHITPSYNYAPNPDKHWIMRYTGPMKPIHMEFTNMLQRKRLQTLMSVDDSMETIYNMLVETGELDNTYIVYTADHGYHIGQFGLVKGKSMPYEFDIRVPFYVRGPNVEAGCLNPHIVLNIDLAPTILDIAGLDIPADMDGKSILKLLDTERPVNRFHLKKKMRVWRDSFLVERGKLLHKRDNDKVDAQEENFLPKYQRVKDLCQRAEYQTACEQLGQKWQCVEDATGKLKLHKCKGPMRLGGSRALSNLVPKYYGQGSEACTCDSGDYKLSLAGRRKKLFKKKYKASYVRSRSIRSVAIEVDGRVYHVGLGDAAQPRNLTKRHWPGAPEDQDDKDGGDFSGTGGLPDYSAANPIKVTHRCYILENDTVQCDLDLYKSLQAWKDHKLHIDHEIETLQNKIKNLREVRGHLKKKRPEECDCHKISYHTQHKGRLKHRGSSLHPFRKGLQEKDKVWLLREQKRKKKLRKLLKRLQNNDTCSMPGLTCFTHDNQHWQTAPFWTLGPFCACTSANNNTYWCMRTINETHNFLFCEFATGFLEYFDLNTDPYQLMNAVNTLDRDVLNQLHVQLMELRSCKGYKQCNPRTRNMDLGLKDGGSYEQYRQFQRRKWPEMKRPSSKSLGQLWEGWEG

SEQ ID NO: 5 - DNA, 21 bp, Artificial Sequence
  Primer
attggaccaaatacaatgaag

SEQ ID NO: 6 - DNA, 45 bp, Artificial Sequence
  Primer
ttaagccttgtcatcgtccttgtagtcgaatgtatcacgccaaat

SEQ ID NO: 7 - DNA, 19 bp, Artificial Sequence
  Primer
cgtgatacattcctagtgg

SEQ ID NO: 8 - DNA, 45 bp, Artificial Sequence
  Primer
ttaagccttgtcatcgtccttgtagtcaccttcccatccatccca

SEQ ID NO: 9 - DNA, 19 bp, Artificial Sequence
  Primer
ccaccttcatcaatgcctt

SEQ ID NO: 10 - DNA, 21 bp, Artificial Sequence
  Primer
ccttgaccagtccaaacctgc

SEQ ID NO: 11 - DNA, 20 bp, Artificial Sequence
  Primer
catcatttacaccgccgacc

SEQ ID NO: 12 - DNA, 19 bp, Artificial Sequence
  Primer
ctgccgtctcttctccttc

SEQ ID NO: 13 - DNA, 21 bp, Artificial Sequence
  Primer
gagccatcttcacccattcaa

SEQ ID NO: 14 - DNA, 22 bp, Artificial Sequence
  Primer
ttcccaaccttatgccttgggt

SEQ ID NO: 15 - DNA, 20 bp, Artificial Sequence
  Primer
accacagtccatgccatcac

SEQ ID NO: 16 - DNA, 21 bp, Artificial Sequence
  Primer
tccaccaccctgttgcttgta

SEQ ID NO: 17 - protein, 24 aa, Homo sapiens
MKYSCCALVLAVLGTELLGSLCST

SEQ ID NO: 18 - DNA, 20 bp, Artificial Sequence
  Primer
catctccatgtctgaacttc

SEQ ID NO: 19 - DNA, 20 bp, Artificial Sequence
  Primer
acctcttccttcaacctctg

SEQ ID NO: 20 - DNA, 21 bp, Artificial Sequence
  Primer
gtcccttgtaatgataataag

SEQ ID NO: 21 - DNA, 18 bp, Artificial Sequence
  Primer
gaagaccaaagtggcatc

SEQ ID NO: 22 - DNA, 21 bp, Artificial Sequence
  Primer
gagtaagaagagatattggag

SEQ ID NO: 23 - DNA, 21 bp, Artificial Sequence
  Primer
cctagctgtgtggatcattgc

SEQ ID NO: 24 - DNA, 20 bp, Artificial Sequence
  Primer
cgaactcctgacctcaagtg

SEQ ID NO: 25 - DNA, 20 bp, Artificial Sequence
  Primer
cagagggtgggtgcagagtc

SEQ ID NO: 26 - DNA, 21 bp, Artificial Sequence
  Primer
tagaatacctgcacttcactg

SEQ ID NO: 27 - DNA, 18 bp, Artificial Sequence
  Primer
gaagaccaaagtggcatc

SEQ ID NO: 28 - protein, 8 aa, Artificial Sequence
  Consensus sequence
XBBBXXBX

SEQ ID NO: 29 - protein, 6 aa, Artificial Sequence
  Consensus sequence
XBBXBX

SEQ ID NO: 30 - protein, 6 aa, Homo sapiens
NKKAKI

SEQ ID NO: 31 - protein, 9 aa, Homo sapiens
LRKKEESSK

SEQ ID NO: 32 - protein, 6 aa, Homo sapiens
LKRRKP

SEQ ID NO: 33 - protein, 8 aa, Homo sapiens
VKKQEKLK

SEQ ID NO: 34 - protein, 16 aa, Homo sapiens
RRRKKERKEKRRQRKG

SEQ ID NO: 35 - DNA, 19 bp, Artificial Sequence
  Primer
aatgctgcccatccacatg

SEQ ID NO: 36 - DNA, 25 bp, Artificial Sequence
  Primer
cagaatcatccactgacatcaaagt

SEQ ID NO: 37 - DNA, 20 bp, Artificial Sequence
  Primer
tcgcaaaacttatgtgaccc

SEQ ID NO: 38 - DNA, 16 bp, Artificial Sequence
  Primer
tccagcacccccaatc

SEQ ID NO: 39 - DNA, 20 bp, Artificial Sequence
  Primer
ccacctaccactgtccgagt

SEQ ID NO: 40 - DNA, 20 bp, Artificial Sequence
  Primer
tctgccgtctcttctccttc

SEQ ID NO: 41 - DNA, 22 bp, Artificial Sequence
  Primer
gagccatcttcacccattcaag

(19) United States
(12) Patent Application Publication
(10) Pub. No.: US 2006/0100153 A1
(43) Pub. Date: May 11, 2006

(54) HSulf-1 nucleic acids, polypeptides and methods of using

(75) Inventors: Viji Shridhar, Rochester, MN (US); Lewis R. Roberts, Rochester, MN (US); Scott H. Kaufmann, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, a Minnesota corporation

(21) Appl. No.: 11/316,132

(22) Filed: Dec. 22, 2005

Related U.S. Application Data
(62) Division of application No. 10/778,607, filed on Feb. 12, 2004.
(60) Provisional application No. 60/446,945, filed on Feb. 12, 2003.

(51) Int. Cl. A61K 38/17; C07K 14/82; C12Q 1/68; G01N 33/574; C07H 21/04; C12P 21/06; A61K 48/00

(52) U.S. Cl. 514/12; 435/6; 435/69.1; 435/7.23; 435/320.1; 435/325; 530/350; 536/23.5; 514/44

(57) ABSTRACT

HSulf-1 nucleic acids and polypeptides are provided, as are methods of using the nucleic acids and polypeptides.

### CROSS-REFERENCE TO RELATED APPLICATIONS

This application claims priority from U.S. Provisional Application Ser. No. 60/446,945, filed Feb. 12, 2003.

### STATEMENT AS TO FEDERALLY SPONSORED RESEARCH

Funding for the work described herein was provided in part by the Department of Defense, grant numbers DAMD17-98-1 and DAMD17-99-1-9504. The federal government may have certain rights in the invention.

### TECHNICAL FIELD

This invention relates to the HSulf-1 nucleic acids and proteins, and to methods for using the nucleic acids and proteins to treat ovarian cancer patients and to detect cancer recurrence in ovarian cancer patients.

### BACKGROUND

Each year in the United States, 27,000 women are diagnosed with ovarian cancer (OvCa) resulting in approximately 14,000 fatalities (Shridhar et al. (2001) Cancer Res. 61:5895-5904). Hepatocellular carcinoma (HCC) is the third leading cause of cancer death worldwide (Ferlay et al. GLOBOCAN 2000: Cancer Incidence, Mortality and Prevalence Worldwide, Version 1.0. 1.0 ed. Lyon: IARCPress, 2001). Because of frequent de novo and acquired resistance of HCCs to chemotherapy, there are as yet no effective chemotherapy regimens for treatment of HCC. In addition, head and neck squamous cell carcinoma (SCCHN) represents about 6% of all new cancers in the United States (Chikamatsu et al. (1999) Int. J. Cancer 82:532-537; Hughes and Frenkel (1997) Am. J. Clin. Oncol. 20:449-461; and Khurana et al. (2001) Head Neck 23:899-906). Despite changes in treatment strategies, prognosis for SCCHN patients has not improved significantly in more than 30 years, with the 5-year survival remaining at 50-60%. Increased understanding of genetic alterations associated with such cancers, as well as the functional consequences of such alterations in cancer would provide groundwork for development of early detection markers, novel therapeutic targets, and better management of diseases such as OvCa, HCC, and SCCHN.

### SUMMARY

The invention is based on the discovery that the gene encoding HSulf-1 is down regulated in tumor cells (e.g., OvCa, HCC, and SCCHN cells). HSulf-1 is a member of an evolutionarily conserved family of proteins analogous to heparan-specific N-acetyl glucosamine sulfatases. The sulfation states of cell surface heparan sulfate proteoglycans (HSPGs) determine both developmental and growth factor signaling. HSulf-1 may be useful for treating cancer patients, as increased expression of HSulf-1 results in induced apoptosis, diminished levels of HSPG sulfation, and a consequent attenuation of growth factor signaling mediated by FGF and HB-EGF. Furthermore, HSulf-1 may enhance the effects of chemotherapeutic agents such as staurosporine, taxol, and cisplatin. Thus, HSulf-1 levels can be used to indicate how well a tumor may respond to treatment with such agents.

In one aspect, the invention features a vector containing an isolated nucleic acid encoding a polypeptide having the amino acid sequence set forth in SEQ ID NO:1 or a fragment thereof.

The invention also features a vector containing an isolated nucleic acid encoding an HSulf-1 polypeptide, wherein the amino acid sequence of the HSulf-1 polypeptide contains a variant relative to the amino acid sequence set forth in SEQ ID NO:1.

In another aspect, the invention features a method for killing a tumor cell. The method can include administering to the tumor cell a nucleic acid that encodes an HSulf-1 polypeptide. The HSulf-1 polypeptide can have the amino acid sequence set forth in SEQ ID NO:1 or a fragment thereof. The amino acid sequence of the HSulf-1 polypeptide can include a variant relative to the amino acid sequence set forth in SEQ ID NO:1. A vector containing the nucleic acid can be administered to the tumor cell.

In another aspect, the invention features a method for killing a tumor cell. The method can include administering to the tumor cell a purified HSulf-1 polypeptide. The HSulf-1 polypeptide can have the amino acid sequence set forth in SEQ ID NO:1 or a fragment thereof. The amino acid sequence of the HSulf-1 polypeptide can contain a variant relative to the amino acid sequence set forth in SEQ ID NO:1.

The invention also features a method for determining the predisposition of an individual to develop cancer. The method can include measuring the level of HSulf-1 polypeptide in a biological sample from the individual. The individual can be predisposed to develop cancer if the level of HSulf-1 polypeptide in the biological sample is lower than the level of HSulf-1 polypeptide in a biological sample from a normal individual. The cancer can be selected from the group consisting of ovarian cancer, hepatocellular carcinoma, head and neck squamous cell carcinoma, breast cancer, and pancreatic cancer.

In still another aspect, the invention features a method for determining whether a tumor will respond to treatment with a chemotherapeutic agent. The method can include determining the level of HSulf-1 mRNA or polypeptide in the tumor. The chemotherapeutic agent can be staurosporine, cisplatin, gemcitabine, topotecan, doxorubicin, or taxol. The HSulf-1 mRNA level can be measured by reverse transcriptase PCR or light cycler PCR. The HSulf-1 polypeptide level can be measured by antibody screening. The tumor can be an ovarian tumor, a liver tumor, a squamous cell tumor, a breast tumor, or a pancreatic tumor.

In another aspect, the invention features a method for detecting-cancer recurrence in an individual diagnosed with and treated for cancer. The method can include measuring the level of HSulf-1 methylation in cells from the individual. The presence of hypermethylation can indicate cancer recurrence, and the absence of hypermethylation can indicates that cancer has not recurred. The cancer can be selected from the group consisting of ovarian cancer, hepatocellular carcinoma, head and neck squamous cell carcinoma, breast cancer, and pancreatic cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic representation of the structure of the gene encoding HSulf-1. Numbered boxes indicate exons; horizontal lines indicate introns. The coding exons (exons 5 through part of exon 23) are indicated by black boxes. The sulfatase domain spans exons 5-13.

FIG. 2 is an alignment of the amino acid sequences of HSulf-1 (SEQ ID NO:1), rat Sulf (SEQ ID NO:2), quail Sulf (Qsulf; SEQ ID NO:3), and KIAA1247 (SEQ ID NO:4). Potential key residue cysteines in the active site of the enzyme are boxed.

FIG. 3 is a schematic of the HSulf-1 open reading frame, showing that the HSulf-1 gene encodes a protein that is 871 amino acid in length and contains a 22 amino acid signal peptide and a 410 amino acid sulfatase domain at the N terminus.

FIG. 4 is a graph showing the percent loss of heterozygosity (LOH) with microsatellite repeats in the introns of HSulf-1 in 33 matched normal/tumor tissue samples.

FIG. 5 is a graph plotting the percent apoptosis rate in SKOV3 parental, vector, and high and low expressing HSulf-1 transfectant clones #6 and #3 after treatment with staurosporine, staurosporine plus a caspase inhibitor, or untreated as indicated.

FIG. 6 is a graph plotting the percent apoptosis in HSulf-1 stable clones 7, 8, and 9 after treatment with 1 μM staurosporine or 7-hydroxystaurosporine (UCN-01). Group 1: untreated cells; Group 2: treated with 1 μM UCN-01; Group 3: treated with 1 μM staurosporine.

FIG. 7 is a graph showing apoptosis levels in SKOV3 cells transfected with expression vectors containing N-Sulf or C-Sulf and treated with staurosporine or UCN-01.

FIG. 8 is a graph plotting apoptosis levels in SKOV3 cells transfected with expression vectors containing wild-type N-Sulf or mutated (CC87, 88AA) N-Sulf, or with empty vector, and treated with 1 μM staurosporine or left untreated.

FIG. 9 is a graph showing apoptosis levels in cells stably transfected with HSulf-1 (stable clone #6) and transiently transfected with antisense or vector constructs, and treated with staurosporine or left untreated.

FIG. 10 is a graph showing hSulf1 activity in parental SNU449 (449) cells, stably transfected SNU449 Vector (Vector) cells, and in stably transfected SNU449 hSulf1-1 (hSulf1-1), SNU182 (182), and SNU475 (475) cells.

FIG. 11A and FIG. 11B are graphs showing FGF-mediated proliferation of SNU449 and Huh-7 cells stably transfected with empty vector or with an HSulf-1 expression vector, and treated with or without FGF as indicated. FIGS. 11C and 11D are graphs showing viability of SNU449 and Huh-7 cells stably transfected with empty vector or with an HSulf-1 expression vector, and treated with or without FGF as indicated.

FIG. 12A is a graph showing the level of apoptosis in parental SNLJ182 (182), SNU475 (475), and SNU449 (449) cell lines, as well as in and stably-transfected SNU449-Vector (Vector) and SNU449-hSulf1 (hSulf1-1, hSulf1-2, and hSulf1-3) cell lines treated with or without staurosporine or with Z-VAD(O-Me)-fmk and staurosporine, as indicated. FIG. 12B is a graph showing the level of apoptosis in the indicated parental and transfected cell lines that were untreated or treated with cisplatin. FIGS. 12C and 12D are graphs showing the level of apoptosis in Vector and hSulf1-transfected stable cell lines derived from the HCC lines Huh-7 (FIG. 12C) and Hep3B (FIG. 12D) treated with or without cisplatin.

FIG. 13A is a graph showing the level of apoptosis in SNU449-hSulf1-1 (hSulf1-1), SNU182, and SNU475 cells transfected with an antisense hSulf1 plasmid or empty vector, and treated with or without staurosporine, as indicated. FIG. 13B is a graph showing the level of apoptosis in SNU449 cells transiently transfected with full-length hSulf1, hSulf1-ΔC, or hSulf1-ΔN-expression and treated with or without staurosporine. FIG. 13C is a graph showing the level of apoptosis in SNU449 cells that wer transiently transfected with empty vector (Vector), a wild-type hSulf1-ΔC-expressing plasmid (hSulf1-ΔC), or a mutant hSulf1-ΔC plasmid with the active-site cysteines in the sulfatase domain replaced by alanines (hSulf1-ΔC-mut), and treated with or without staurosporine.

FIG. 14 is a graph showing the level of sulfatase activity in cell extracts from stable squamous cell carcinoma (012SCC) clones.

FIG. 15 is a graph showing DNA synthesis in 012SCC cells transfected with vector or an HSulf-1 expression vector.

FIGS. 16A and 16B are graphs showing the level of apoptosis in the indicated 012SCC (FIG. 16A) and WMMSCC (FIG. 16B) cell lines after treatment with or without staurosporine.

### DETAILED DESCRIPTION

In general, the invention provides materials and methods related to killing a tumor cell (e.g., an OvCa cell, a HCC cell, a SCCHN cell, a breast cancer cell, or a pancreatic cancer cell), and for determining predisposition to or treatability of cancer (e.g., OvCa, HCC, SCCHN, breast cancer, or pancreatic cancer) in an individual. In particular, the invention provides materials and methods related to HSulf-1, a gene that is down regulated in cancer cells (e.g., OvCa, HCC, and SCCHN cells). HSulf-1 may be useful for treating cancer patients, as increased expression of HSulf-1 results in stress-induced apoptosis, diminished levels of HSPG sulfation, and a consequent attenuation of growth factor signaling mediated by FGF and HB-EGF. Furthermore, HSulf-1 may enhance the effects of chemotherapeutic agents such as staurosporine, taxol, and cisplatin. Thus, HSulf-1 levels can be used to indicate how well a tumor may respond to treatment with such agents.

Isolated HSulf-1 Nucleic Acid Molecules

The invention provides isolated HSulf-1 nucleic acid molecules. Such nucleic acids can contain all or part of the coding sequence and/or non-coding sequence from the HSulf-1 gene. As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand). As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that flank an HSulf-1 gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one or both of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nutcleic acid.

Isolated nucleic acid molecules are at least 10 nucleotides in length (e.g., 10, 20, 50, 100, 200, 300, 400, 500, 1000, or more than 1000 nucleotides in length). As described in the Examples herein, the full-length human HSulf-1 transcript contains 23 exons, with a coding region that is 2613 nucleotides in length. The full-length HSulf-1 sequence also is provided in GenBank (Accession No. AF545571). An HSulf-1 nucleic acid molecule is not required to contain all of the coding region or all of the exons; in fact, an HSulf-1 nucleic acid molecule can contain as little as a single exon or a portion of a single exon (e.g., 10 nucleotides from a single exon). Nucleic acid molecules that are less than full-length can be useful, for example, for diagnostic purposes.

Isolated nucleic acid molecules of the invention can be produced by standard techniques, including, without limitation, common molecular cloning and chemical nucleic acid synthesis techniques. For example, polymerase chain reaction (PCR) techniques can be used to obtain an isolated HSulf-1 nucleic acid molecule. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis (1992) Genetic Engineering News 12(9): 1; Guatelli et al. (1990) Proc. Natl. Acad. Sci. USA 87:1874-1878; and Weiss (1991) Science 254:1292-1293.

Isolated HSulf-1 nucleic acid molecules also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Vectors and Host Cells

The invention also provides vectors containing nucleic acids such as those described herein. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence.

In the expression vectors of the invention, the nucleic acid is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as glutathione S-transferase (GST), polyhistidine, c-myc, hemagglutinin, or FLAG® tag (Kodak, New Haven, Conn.) sequences typically are expressed as a fusion with the encoded polypeptide. Such tags can be inserted anywhere within the polypeptide including at either the carboxyl or amino terminus.

The invention also provides host cells containing vectors of the invention. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., LIPOFECTIN® (Invitrogen/Life Technologies); FUGENE™ (Roche, Indianapolis, Ind.); and SUPERFECT® (Qiagen, Valencia, Calif.)).

Purified HSulf-1 Polypeptides

The invention provides purified HSulf-1 polypeptides that are encoded by the HSulf-1 nucleic acid molecules described herein. A “polypeptide” refers to a chain of at least 10 amino acid residues (e.g., 10, 20, 50, 75, 100, 200, 300, 400, 500, 600, 700, 800, or more than 800 residues), regardless of post-translational modification (e.g., phosphorylation or glycosylation). Typically, an HSulf-1 polypeptide of the invention is capable of eliciting an HSulf-1-specific antibody response (i.e., is able to act as an immunogen that induces the production of antibodies capable of specific binding to HSulf-1 polypeptide).

An HSulf-1 polypeptide may contain an amino acid sequence that is identical to at least a portion of SEQ ID NO:1. Alternatively, an HSulf-1 polypeptide can include an amino acid sequence variant. As used herein, an amino acid sequence variant refers to a deletion, insertion, or substitution with respect to the reference amino acid sequence set forth in SEQ ID NO:1. For example, an HSulf-1 polypeptide can contain amino acid substitutions at up to twenty amino acid positions (e.g., one, two, three, four, five, six, seven, eight, nine, ten, 11, 12, 13, 14, 15, 16, 17, 18, 19, or 20 positions) relative to the full-length amino acid sequence set forth in SEQ ID NO:1. In another embodiment, an HSulf-1 polypeptide can be a fusion polypeptide that contains an HSulf-1 amino acid sequence linked to an amino acid tag (e.g., FLAG®, His, or c-myc), or to another polypeptide such as green fluorescent protein (GFP). In yet another embodiment, an HSulf-1 polypeptide can contain an amino acid sequence that is a fragment of that set forth in SEQ ID NO:1. A fragment can contain, for example, from about 50 to about 850 amino acid residues (e.g., about 50, 75, 100, 150, 200, 300, 400, 500, 600, 700, 800, or about 850 amino acid residues).

The term “purified” as used herein with reference to a polypeptide refers to a polypeptide that either has no naturally occurring counterpart (e.g., a peptidomimetic), has been chemically synthesized and is thus uncontaminated by other polypeptides, or has been separated or purified from other cellular components by which it is naturally accompanied (e.g., other cellular proteins, polynucleotides, or cellular components). Typically, a polypeptide is considered “purified” when it is at least 70% (e.g., 70%, 80%, 90%, 95%, or 99%), by dry weight, free from the proteins and naturally occurring organic molecules with which it naturally associates.

HSulf-1 polypeptides can be produced by a number of methods, many of which are well known in the art. By way of example and not limitation, HSulf-1 polypeptides can be obtained by extraction from a natural source (e.g., from isolated cells, tissues or bodily fluids), by expression of a recombinant nucleic acid encoding the polypeptide, or by chemical synthesis.

HSulf-1 polypeptides of the invention can be produced by, for example, standard recombinant technology, using expression vectors encoding HSulf-1 polypeptides. The resulting HSulf-1 polypeptides then can be purified. Expression systems that can be used for small or large scale production of HSulf-1 polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules of the invention; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids of the invention.

Suitable methods for purifying the HSulf-1 polypeptides provided herein can include, for example, affinity chromatography, immunoprecipitation, size exclusion chromatography, and ion exchange chromatography. See, for example, Flohe et al. (1970) Biochim. Biophys. Acta. 220:469-476; and Tilgmann et al. (1990) FEBS 264:95-99. The extent of purification can be measured by any appropriate method, including but not limited to column chromatography, polyacrylamide gel electrophoresis, and high-performance liquid chromatography. HSulf-1 polypeptides also can be “engineered” to contain a tag sequence as described herein that allows the polypeptide to be purified (e.g., captured onto an affinity matrix). Immunoaffinity chromatography also can be used to purify HSulf-1 polypeptides.

Methods of Using HSulf1 Nucleic Acids and Polypeptides

An HSulf-1 nucleic acid or polypeptide can be used in the manufacture of a medicament for treating cancer. In addition, the invention provides methods for using HSulf-1 nucleic acid molecules and polypeptides to treat individuals with cancer (e.g., OvCa, HCC, SCCHN, breast cancer, or pancreatic cancer). For example, a vector containing an HSulf-1 nucleic acid sequence that encodes an HSulf-1 polypeptide can be administered to a tumor cell, such that expression of the encoded HSulf-1 polypeptide can induce apoptosis and kill the tumor cell. Suitable methods for introducing nucleic acids into cells include those known in the art, such as the transfection and transformation techniques disclosed herein. Any other suitable method of transferring a nucleic acid molecule into a cell (e.g., viral transformation) also can be used.

In another embodiment, an HSulf-1 polypeptide (e.g., an HSulf1 polypeptide containing the amino acid sequence of SEQ ID NO:1 or a fragment thereof) can-be administered directly to a tumor cell (e.g., an OvCa, HCC, or SCCHN tumor cell in a mammal such as a human) in order to kill the cell. In some embodiments, implantable medical devices can be used to deliver HSulf-1 polypeptides to a mammal, and in particular to a human patient. For example, HSulf-1 polypeptides can be incorporated into a coated device such that the polypeptides are eluted over time. Alternatively, a medical device can be seeded with cells such as smooth muscle cells, fibroblasts, hepatocytes, endothelial cells, epithelial cells, or stem cells in vitro, and then implanted into a patient. Typically, cells are harvested from the patient in whom the medical device will be implanted. In some embodiments, however, cells can be harvested from a donor of the same or of a different species that is not the recipient of the medical device. For example, it may be useful to harvest cells from a pig for transplantation into a human. Cells that are seeded onto the medical device can be modified such that the cells produce HSulf-1 polypeptides. Such polypeptides can be secreted into the vasculature, for example. Implantable medical devices thus can deliver an HSulf-1 polypeptide to a mammal for treating a cancer such as, for example, OvCa, HCC, SCCHN, breast cancer, or pancreatic cancer.

To modify isolated cells such that an HSulf-1 polypeptide is produced, an appropriate exogenous nucleic acid can be delivered to the cells. Primary cultures or secondary cell cultures can be modified and then seeded onto an implantable device. In some embodiments, transient transformants in which the exogenous nucleic acid is episomal (i.e., not integrated into the chromosomal DNA), can be seeded onto a medical device. Typically, stable transformants in which the exogenous nucleic acid has integrated into the host cell's chromosomal DNA are selected. The term “exogenous” as used herein with reference to a nucleic acid and a particular cell refers to any nucleic acid that does not originate from that particular cell as found in nature. In addition, the term “exogenous” includes a naturally occurring nucleic acid. For example, a nucleic acid encoding a polypeptide that is isolated from a human cell is an exogenous nucleic acid with respect to a second human cell once that nucleic acid is introduced into the second human cell.

An exogenous nucleic acid can be transferred to cells within a primary or secondary culture using recombinant viruses that can infect cells, or liposomes or other non-viral methods such as electroporation, microinjection, or calcium phosphate precipitation. The exogenous nucleic acid that is delivered typically is part of a vector in which a regulatory element such as a promoter is operably linked to the nucleic acid of interest. The promoter can be constitutive or inducible. Non-limiting examples of constitutive promoters include the cytomegalovirus (CMV) promoter and the Rous sarcoma virus (RSV) promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus. The inducer also can be an illumination agent such as light and light's various aspects, which include wavelength, intensity, fluorescence, direction, and duration.

An example of an inducible promoter is the tetracycline (tet)-on promoter system, which can be used to regulate transcription of the nucleic acid. In this system, a mutated Tet repressor (TetR) is fused to the activation domain of herpes simplex VP16 (transactivator protein) to create a tetracycline-controlled transcriptional activator (tTA), which is regulated by tet or doxycycline (dox). Transcription is minimal in the absence of antibiotic, while transcription is induced in the presence of tet or dox. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A.

Additional regulatory elements that may be useful in vectors include, without limitation, polyadenylation sequences, translation control sequences (e.g., an internal ribosome entry segment, IRES), enhancers, and introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, can sometimes be obtained without such additional elements.

Other elements that can be included in vectors include nucleic acids encoding selectable markers. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthinb-guanine phosphoribosyltransferase (XGPRT). Such markers are useful for selecting stable transformants in culture.

Viral vectors also can be used to introduce an exogenous nucleic acid into a cell. Suitable viral vectors include, for example, adenovirus, adeno-associated virus (AAV), retroviruses, lentiviruses, vaccinia virus, measles viruses, herpes viruses, and bovine papilloma virus vectors. See, Kay et al. (1997) Proc. Natl. Acad. Sci. USA 94:12744-12746 for a review of viral and non-viral vectors. Viral vectors are modified so the native tropism and pathogenicity of the virus has been altered or removed. The genome of a virus also can be modified to increase its infectivity and to accommodate packaging of the nucleic acid encoding the polypeptide of interest.

Adenoviral vectors can be easily manipulated in the laboratory, can efficiently transduce dividing and nondividing cells, and rarely integrate into the host genome. Smith et al. (1993) Nat. Genet. 5:397-402; and Spector and Samaniego (1995) Meth. Mol. Genet. 7:31-44. The adenovirus can be modified such that the E1 region is removed from the double stranded DNA genome to provide space for the nucleic acid encoding the polypeptide and to remove the transactivating E1a protein such that the virus cannot replicate. Adenoviruses have been used to transduce a variety of cell types, including, inter alia, keratinocytes, hepatocytes, and epithelial cells.

Adeno-associated viral (AAV) vectors demonstrate a broad range of tropism and infectivity, although they exhibit no human pathogenicity and do not elicit an inflammatory response. AAV vectors exhibit site-specific integration and can infect non-dividing cells. AAV vectors have been used to deliver nucleic acid to brain, skeletal muscle, and liver over a long period of time (e.g., greater than 9 months in mice) in animals. See, for example, U.S. Pat. No. 5,139,941 for a description of AAV vectors.

Retroviruses are the most-characterized viral delivery system and have been used in clinical trials. Retroviral vectors mediate high nucleic acid transfer efficiency and expression. Retroviruses enter a cell by direct fusion to the plasma membrane and integrate into the host chromosome during cell division.

Lentiviruses also can be used to deliver nucleic acids to cells, and in particular, to non-dividing cells. Replication deficient HIV type I based vectors have been used to transduce a variety of cell types, including stem cells. See, Uchidda et al. (1998) Proc. Natl. Acad. Sci. USA 95:11939-11944.

Non-viral vectors can be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased using dioleoylphosphatidyl ethanolamine during transduction. See, Felgner et al. (1994) J. Biol. Chem. 269:2550-2561. High efficiency liposomes are commercially available. See, for example, SUPERFECT® from Qiagen.

In another embodiment, the invention provides methods for determining whether an individual is predisposed to develop cancer (e.g., OvCa, HCC, SCCHN, breast cancer, or pancreatic cancer). A method can involve, for example, measuring the amount of HSulf-1 mRNA or protein in a biological sample (e.g., blood, ovarian cells, liver cells, or epithelial cells) obtained from an individual, and comparing the amount of mRNA or protein to, for example, an amount of HSulf-1 mRNA or protein determined from an individual known to have a cancer such as OvCa, HCC, SCCHN breast cancer, or pancreatic cancer, an individual who does not have such a cancer, or an average amount determined from measuring HSulf-1 mRNA or protein in a population of individuals that have or do not have such a cancer.

As used herein, a biological sample contains cells or cellular material, and can include, for example, urine, blood, cerebrospinal fluid, pleural fluid, sputum, peritoneal fluid, bladder washings, secretions, oral washings, tissue samples, touch preps, or fine-needle aspirates. Since HSulf-1 is a secreted protein, the amount of HSulf-1 in a blood sample obtained from an individual can be used to determine whether that individual is predisposed to cancer (e.g., OvCa, HCC, or SCCHN). If the amount of HSulf-1 mRNA or protein in, for example, ovarian cells, liver cells, epithelial cells, or blood from the individual is lower than the amount in cells obtained from a normal individual (i.e., an individual who does not have cancer), then the individual in question may be predisposed to develop cancer such as, for example, OvCa, HCC, or SCCHN. Alternatively, if the amount HSulf-1 mRNA or protein in ovarian cells or blood from the individual is higher than the amount in cells obtained from a cancer patient, then the individual in question may not be predisposed to develop cancer.

In addition, the invention provides methods for classifying tumors as chemotherapy responders or non-responders based on the level of HSulf-1 present in the tumors. As described herein (see, e.g., Examples 8 and 9), HSulf-1 expression in tumor cells can reduce growth factor signaling. Thus, tumor cells that express HSulf-1 may be more sensitive to apoptosis-inducing chemotherapeutic agents. For example, a tumor that contains HSulf-1 polypeptides may respond better to treatment with chemotherapeutic agents such as staurosporine, cisplatin, taxol, topotecan, gemcitabine, or doxorubicin, while a tumor that contains little or no HSulf-1 may not respond well to such treatment. Levels of HSulf-1 in a tumor can be measured using techniques such as reverse transcriptase PCR (RT-PCR) or light cycler PCR with RNA obtained from tumor cells, or immuno-screening of tumor cells with an anti-HSulf-1 antibody.

The invention also provides methods for detecting cancer recurrence in an individual (e.g., an individual with OvCa, HCC, SCCHN, breast cancer, or pancreatic cancer). Methods can include measuring the level of methylation of the HSulf-1 gene in cells obtained from a cancer patient, and comparing the level to a standard level of methylation (e.g., the level of HSulf-1 methylation in cells obtained from a normal individual who does not have cancer). For example, cells can be obtained from a peritoneal washing of an individual who has been treated for cancer (e.g., an individual treated for OvCa or HCC undergoing second look laparoscopy or laparotomy (SLL)), and the degree of HSulf-1 hypermethylation can be determined as discussed in Example 14, for example. As used herein, “hypermethylation” means that the HSulf-1 gene is more highly methylated in the test individual than in a normal individual. The presence or absence of hypermethylation can be used as an indicator of the presence or absence, respectively, of cancer cells in an individual (e.g., a patient having OvCa, HCC, SCCHN, breast cancer, or pancreatic cancer). In turn, the presence or absence of cancer cells in the individual can indicate whether or not cancer has recurred.

The invention will be further described in the following examples, which do not limit the scope of the invention described in the claims.

### EXAMPLES

### Example 1

### Materials and Methods for OvCa Studies

Cell Culture: The ovarian-carcinoma cell lines OV167, OV177, OV202, OV207, and OV266 were established at the Mayo Clinic (Conover et al. (1998) Exp. Cell Res. 238:439-449). Two addition ovarian-carcinoma cell lines, OVCAR-5 and SKOV-3, were purchased from the American Type Culture Collection (ATCC; Manassas, Va.). All cells were grown according to the provider's recommendations.

Drugs and Reagents: Staurosporine (Sigma, St. Louis, Mo.) and UCN-01 (Drug Synthesis Branch, National Cancer Institute) were dissolved in DMSO at a concentration of 1 mM, stored at −20° C. and subsequently diluted with serum free medium before use. In all experiments the concentration of DMSO did not exceed 0.1%. The broad-spectrum caspase inhibitor N-(N^{α}-benzyloxycarbonylvalinylalanyl) aspartic acid (O-methyl ester) fluoromethylketone [Z-VAD(OMe)-fmk] was dissolved in DMSO and stored at 4° C.

Strategy for Cloning the Gene: A BLAST search of the isolated sequences from SSH libraries of early and late stage tumors identified ESTs homologous to KIAA1077 in the dbEST. The homologous ESTs were assembled into a contig with using Sequencher 3 (Gene Codes Corp, Ann Arbor, Mich.) software. An additional five sequences not present in KIAA1077 were obtained with electronic walking by assembly of overlapping EST sequences in the genome BLAST server. The integrity of the full-length cDNA obtained by this electronic walking was confirmed by PCR analysis using PCR primers flanking each junction between EST clones. The entire cDNA contig was sequenced twice with overlapping primers.

Cloning of FLAG® (Flg)-tagged N-terminal Sulf, C terminal Sulf and full-length Sulf: The N terminal portion of HSulf-1 (N-Sulf) containing only the sulfatase domain was amplified using primers NF (5′-ATTGGACCAAATACAATGAAG; SEQ ID NO:5) and NRFlg (5′-ttaagccttgtcatcgtccttgtagtcGAATGTATCACGCCAAAT; SEQ ID NO:6). The C terminal domain (C-Sulf) was amplified using primers CF (5′-CGTGATACATTCCTAGTGG; SEQ ID NO:7) and CRFlg (5′-ttaagccttgtcatcgtccttgtagtcACCTTCCCATCCATCCCA; SEQ ID NO:8) with a stop codon introduced after the epitope tag (lower case letters). The N-Sulf and C-Sulf fragments each were about 1350 basepairs in length. The full-length (FL) HSulf-1 was amplified using primers NF and CRFlg using EXPAND™ Long Template PCR system (Boehringer Mannheim, Indianapolis, Ind.). All three products were cloned into GFP Fusion TOPO® TA Expression plasmid (Invitrogen/Life Technologies). To generate a FL HSulf-1 GFP fusion construct for immunocytochemistry, the stop codon of CRFlg was not included. cDNAs generated from short-term cultures of normal ovarian surface epithelial cells (OSE) were used as a template for generating PCR products for cloning. The products of each PCR reaction were resolved on a 1.6% agarose gel and purified using a gel extraction kit (Qiagen) for cloning into expression vectors.

Establishment of HSulf-1-Stable Transfectants: Exponentially growing SKOV3 cells in 100 mm dishes were washed with serum free medium, and treated with a mixture of 4 μg of FL HSulf-1 plasmid, 30 μl of LIPOFECTAMINE™, and 20 μl of PLUS™ reagent. After a 3 hour incubation, complete medium with serum was added. G418 (400 μg/ml) was added 24 hours after transfection to select transfectants. Several stable clonal transfectants, HSulf-1 clones #3-9, were subsequently generated. For controls, cells were similarly transfected with vector (pcDNA3.1 GFP) and selected.

Semi-quantitative RT-PCR: Total RNA was extracted from 7 ovarian cancer cell lines and 31 primary ovarian tumors using the RNEASY® mini kit (Qiagen). cDNA synthesis was performed as described (Shridhar et al. 2002 Cancer Res. 62:262-270). Reverse transcribed cDNA (50-100 ng) was used in a multiplex reaction with three different Sulf primer pairs: Sulf-1F (5′-CCACCTTCATCAATGCCTT; SEQ ID NO:9), Sulf-1R (5′-CCTTGACCAGTCCAAACCTGC; SEQ ID NO:10), Sulf-2F (5′-CATCATTTACACCGCCGACC; SEQ ID NO:11), Sulf-2R (5′-CTGCCGTCTCTTCTCCTTC; SEQ ID NO:12), Sulf-3F (5′-GAGCCATCTTCACCCATTCAA; SEQ ID NO:13), Sulf-3R (5′-TTCCCAACCTTATGCCTTGGGT; SEQ ID NO:14), as well as a control primer pair to GAPDH: GAPDH-F (5′-ACCACAGTCCATGCCATCAC-3; SEQ ID NO:15) and GAPDH-R (5′-TCCACCACCCTGTTGCTTGTA; SEQ ID NO:16) in separate reactions to yield Sulf reaction products of 760 bp, 1260 bp and 825 bp. The PCR reaction mixes contained 50 mM KCl, 10 mM Tris-HCl (pH 8.3), 1.5 mM MgCl₂, 400 μM of each primer for HSulf-1 and 50 μM for the GAPDH primers, and 0.5 units of Taq polymerase (Promega, Madison, Wis.) in a 12.5 μl reaction volume. The conditions for amplification were: 94° C. for 3 minutes followed by 29 cycles of 94° C. for 30 seconds, 58° C. for 30 seconds, and 72° C. for 30 seconds in a Perkin Elmer-Cetus 9600 Gene-Amp PCR system (Perkin Elmer-Cetus, Wellesley, Mass.). The products of the reaction were resolved on a 1.6% agarose gel and quantified using a GEL DOC™ 1000 photo documentation system (Bio-Rad Laboratories, Hercules, Calif.).

LOH Analysis: The 5 pairs of primers used to amplify regions containing microsatellite markers within the HSulf-1 gene are listed in Table I, along with their locations within the HSulf-1 gene. Amplifications were performed as described (Shridhar et al. (2002) supra) except that annealing was performed at 52-57° C. and reactions were run in a 96 well plate. After denaturation, PCR products were run on 6% polyacrylamide sequencing gels containing 8 M urea. Gels were dried, subjected to autoradiography using multiple exposure times, and scored for LOH. Allelic imbalance indicative of LOH was scored when there was more than 50% loss of intensity of one allele in the tumor sample with respect to the matched allele from normal tissue.

Northern Blotting: Total RNA (15 μg) was fractionated on 1.2% formaldehyde agarose gels and blotted in 1×SPC buffer (20 mM Na₂HPO₄, 2 mM 1,2-cyclohexylenediaminetetraacetic acid (CDTA), pH 6.8) onto Hybond-N membranes (Amersham, Piscataway, N.J.). The probes were labeled using the random primer labeling system (Life Technologies, Inc.) and purified using spin columns (100 TE) from Clontech. Filters were hybridized at 68° C. with radioactive probes in a hybridization incubator (Model 2000; Robbins Scientific, Sunnyvale, Calif.) and washed according to the manufacturer's guidelines.

Sulfatase Activity in HSulf-1 Expressing Cells: Cultured cell lines were collected by scraping, washed, and centrifuged. Equal amounts of cells (5×10⁶) were suspended in 2 ml of lysis buffer (10 mM Hepes, 150 mM NaCl, 1% NP-40, 10% glycerol), to which was added a protease inhibitor mixture consisting of 1 mM phenylmethylsulfonyl fluoride, 5 mg/ml chymostatin, leupeptin, aprotinin, pepstatin, and soybean trypsin inhibitor on ice for 10 minutes. Cell lysates were stored at −80° C. before use. Duplicate aliquots (100 μl) of each cell lysate in SIE (86 g sucrose, 10 ml of 300 mM imidazole, 1 ml absolute ethanol, pH 7.4) were kept on ice. Freshly prepared buffered substrate mix (100 μl of a solution containing 2.94 mg 4-methylumbelliferyl-sulfate (4-MUS) in 10 ml DMSO; Sigma Chemicals, St Louis, Mo.) was added into each tube at convenient time intervals (10-15 seconds). The mix was shaken gently and incubated at 37° C. for 20 minutes. Two ml of stopping solution (50 mM glycine containing 5 mM EDTA, pH 10.4) was added at same time intervals and vortexed to mix thoroughly. Sulfatase activity was determined using fluorometry to measure release of 4-methylumbelliferone ( Karpova et al. (1996) J. Inherit. Metab. Dis. 19:278-85) with an excitation wavelength of 360 nm and an emission wavelength of 460 nm. Activity was expressed in nanomoles of released 4-methylumbelliferone per minute per mg of protein.

Analysis of Apoptosis: Apoptosis was quantitated using fluorescence microscopy to assess the nuclear changes indicative of apoptosis (chromatin condensation and nuclear fragmentation), using the DNA binding dye 4,6-diamidino-2-phenylindole (DAPI) dihydrochloride. HSulf-1 transfected SKOV3 cells were seeded in 35-mm plates at a density of 2×10⁵ cells/well. After incubation at 37° C. for 24 hours, the plates were washed and changed to serum-free medium. Staurosporine was added to a final concentration of 1 μm for 5 hours. DAPI was then added to each well. After a 20 minute incubation at room temperature in the dark, cells were examined by fluorescence microscopy (Nikon Eclipse TE200; Nikon Corp., Tokyo, Japan) using excitation and emission filters of 380 and 430 nm. An individual blinded to the experimental conditions counted at least 300 cells in six different high-power fields for each treatment. Each treatment was repeated at least three times in triplicate. To inhibit apoptosis, the cells were pretreated with 40 μM Z-VAD(OMe)-fmk for 1 hour before the addition of staurosporine. The significance of differences between experimental variables was determined using the Student t test.

DNA Fragmentation: Parental, vector and stable HSulf-1 clones 3 and 6 were treated with 1 μM staurosporine at 37° C. for 5 hours. Control or treated cells (5×10⁵ cells) were harvested with trypsin and centrifuged. DNA was extracted using the Qiagen DNEASY® kit. Aliquots containing 5 μg of DNA were resolved on a 1.5% agarose gel containing 0.5 μg/ml ethidium bromide and visualized under UV light.

Flow Cytometry: After treatment with 1 μM staurosporine for 5 hours as described above, cells were washed twice in ice cold PBS containing 3% heat-inactivated fetal bovine serum and 0.02% sodium azide, stained with 7-aminoactinomycin D (7-AAD; 50 μg/ml) for 15 minutes in the dark, resuspended in 500 μl PBS, and subjected to flow cytometry on a FACScan analyzer (BD Bioscience, San Jose, Calif.). After analysis of 10,000 events, the percentage of 7-AAD positive cells was determined.

Analysis of Cytosolic Cytochrome c: Parental SKOV3, stable clones of SKOV3-vector, and SKOV3-HSulf-1 clones 3 and 6 were treated with staurosporine as described above, washed in PBS, and incubated for 30 seconds in lysis buffer consisting of 210 mM D-mannitol, 70 mM sucrose, 10 mM HEPES, 5 mM sodium succinate, 0.2 mM EGTA, 0.15% BSA and 80 μg/ml digitonin. After sedimentation at 12,000×g for 1 minute, the supernatant was diluted with an equal volume of 2×sample buffer. The protein samples were quantified, resolved on a 12% SDS-PAGE gel, and subjected to immunoblot analysis as described below using anti-cytochrome c (mouse monoclonal; BD Pharmingen, San Diego, Calif.) at a dilution of 1:500.

FGF2, HB-EGF and EGF Treatment and Protein Extraction: To confirm the role of HSulf-1 in HB-GF mediated signaling, vector-transfected and HSulf-1 clones 7 and 8 were serum starved for 8-12 hours, treated with diluent, 1 ng/ml FGF2, 100 ng/ml of HB-EGF (Sigma, St Louis, Mo.) or 10 ng/ml EGF. Following treatment, cells were rinsed with ice cold PBS, scraped from the dishes, and lysed at 4° C. in Laemelli buffer without bromophenol blue. Protein concentrations were determined with bicinchonic acid (Pierce, Rockford, Ill.).

Immunoblotting: Equal amounts of protein (20 μg/lane) were separated by electrophoresis on a 4-12% glycine-SDS gel and electrophoretically transferred to nitrocellulose. Blots were washed once with TBS-0.2% Tween 20 (TBST) and blocked with TBST containing 5% non-fat dry milk for 1 hour at room temperature. The blocking solution was replaced with a fresh solution containing 1:500 dilution of rabbit anti-phospho42/44MAPK (Cell Signaling Inc., Beverly, Mass.). After overnight incubation at 4° C., the blots were washed three times for 10 minutes each in TBS/0.1% Tween and incubated with horseradish peroxidase-conjugated secondary antibody in 5% milk/TBST at room temperature for 1 hour. After washing 3 times in TBST, the proteins were visualized using enhanced chemiluminescence (Amersham). The blots were stripped and reprobed with 1:500 dilution of antibody to total MAPK (Cell Signaling Inc.), 1:1000 dilution of rabbit sera that recognize EGFR phosphorylated on Tyr 1068 and/or 992, 1:1000 dilution of rabbit anti EGFR (Cell Signaling Inc.), and/or 1:1000 dilution of mouse monoclonal antibody to actin (Sigma).

HSulf-1 Localization. SKOV3 cells that had been seeded on glass cover slips in 6-well plates and incubated overnight were transfected with C-terminal GFP-tagged full-length HSulf-1 or GFP expression plasmid as a control. Twenty-four hours after transfection, cells were fixed in 4% paraformaldehyde, permeabilized with 0.2% Triton X-100, and mounted with VECTORSHIELD® mounting medium with DAPI. The GFP fusion protein was visualized using a Zeiss LS510 laser scanning confocal microscope. Alternatively, SKOV3 cells were transfected with 4 μg of FLAG®-tagged HSulf-1 construct, incubated for 24 hours, washed with PBS, fixed for 10 minutes in PBS containing 3.7% formaldehyde and 1% sucrose, washed with 0.1 M glycine in PBS, permeabilized with PBS containing 0.4% Triton X-100 and 2% BSA for 20 minutes, and washed three times in washing buffer (PBS containing 0.2% bovine serum albumin and 0.1% Triton X-100). After incubation for 1 hour at room temperature with anti-EGFR antibody, cells were washed four times with washing buffer, incubated with 1:200 TRITC-conjugated anti-rabbit IgG and 1:200 FITC-conjugated anti-FLAG® monoclonal antibody (Sigma) in the dark, washed twice in washing buffer, stained with 0.5 μg/ml DAPI for 5 minutes, washed twice in PBS, mounted onto slides, and viewed with an Axiovert 35 epifluorescence microscope (Carl Zeiss Thornwood, N.Y.) equipped with a 100-W mercury lamp or a confocal microscope (Zeiss LSM-510).

Sulfation State of Cell Surface HS-GAGs: Parental, stable vector, and stable HSulf-1 clones 7, 8, and 9 were grown on cover slips for 24 hours, fixed in methanol for 10 minutes at −20° C., washed with PBS, and incubated for 1 hour at room temperature with a 1:30 dilution of primary anti-mouse antibody recognizing native heparan sulfate that includes the N-sulfated glucosamine residue (10E4-mAb Seikagaku America, Falmouth, Mass.). After washing, cells were stained with FITC-conjugated anti-mouse IgG and examined by laser scanning confocal microscopy as described herein.

### Example 2

### Isolation and Cloning of a Novel cDNA Containing a Conserved Sulfatase Domain

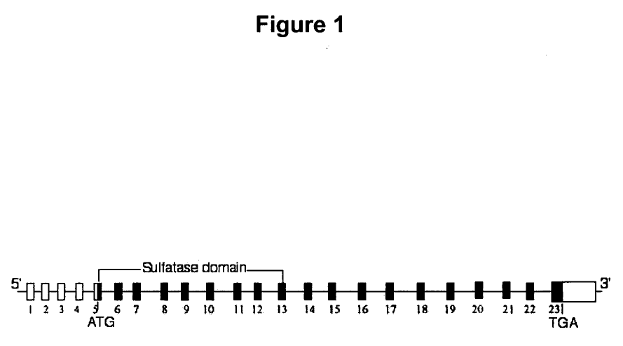

Differential screening of suppression subtraction cDNA libraries generated from primary ovarian tumors subtracted against normal ovarian epithelial cells (Shridhar et al. (2002) supra) identified an EST homologous to KIAA1077 in the GenBank database. Examination of the 4834 bp sequence of KIAA1077 in the database revealed that it was a partial cDNA. Once the full-length HSulf-1 was assembled into a contig with the use of Sequencher 3 software, the full-5699 bp cDNA containing a single open reading frame coding for a 871 amino-acid long protein was isolated (GenBank accession number, AF545571). A putative initiation codon occurs within a strong Kozak context (Kozak (1999) Gene 234:187-208) and is preceded by a stop codon. HSulf-1 was mapped to chromosome 8q13.3 based on the Human Genome BLAST server database. This information, combined with PCR analysis, was used to map the 23 exons of HSulf-1 distributed across approximately 250 kb of DNA. The sulfatase domain (1230 bases) extends from the 3′ end of exon 5 (41 bases) through most of exon 13. The translational codon initiates in exon 5 (FIG. 1). There are 706 bases of 5′ UTR and 2377 bases in the 3′ UTR. There are six potential polyadenylation signals (AATAAA) at positions 4170, 4679, 4820, 4824, 5052 and 5678.

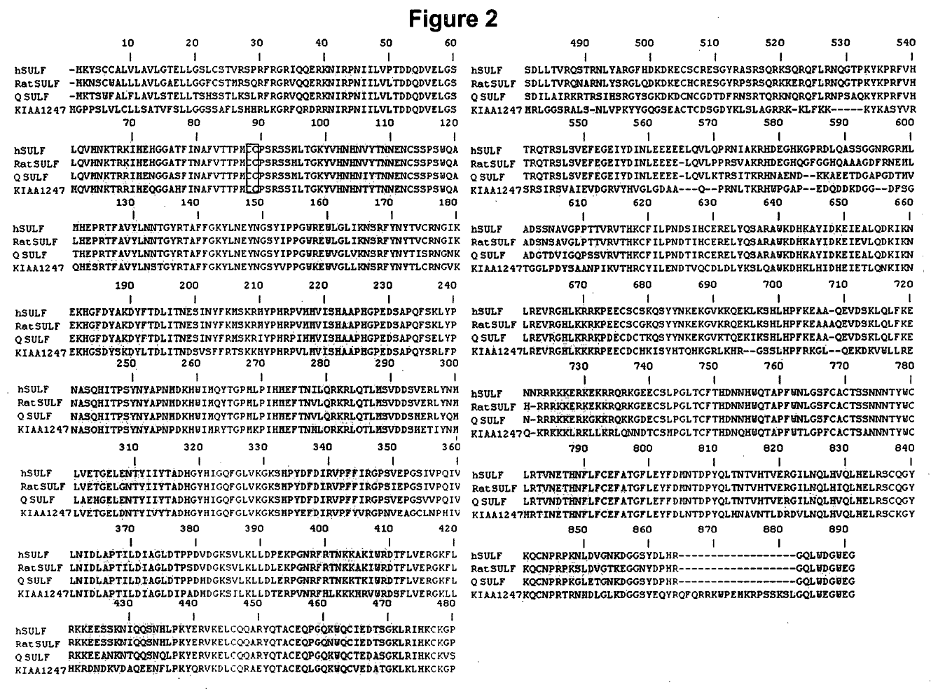

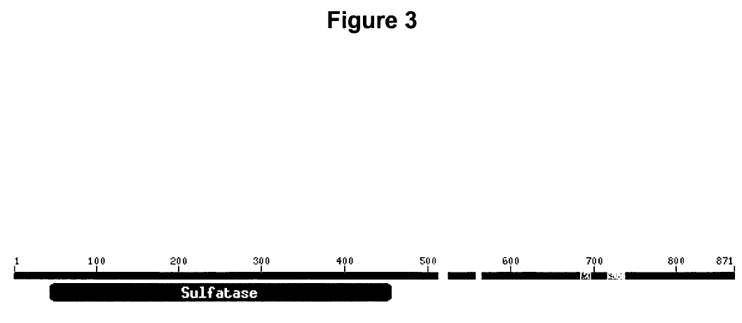

The predicted protein encoded by HSulf-1 (KIAA 1077) shares extensive sequence homology with rat Sulf (88%) and to a recently identified sulfatase protein in quail embryos (Qsulf; 81%; FIG. 2) as well as 30% and 45% identity to two other sulfatase domain containing proteins, arylsulfatase and N-acetylglucosamine-6 sulfatase, respectively. HSulf-1 also is 62% identical to KIAA1247, a gene mapping to 20q12-13. The SULF1 gene of D. melanogaster and C. elegans also have a high degree of identity to HSulf-1 (59% and 50% respectively). Based on the computer algorithm, Signal PV1.1 at the Centre for biological Sequence Analysis website (Nielsen et al. (1997) Int. J. Neural Syst. 8:581-599), a 22 amino acid long N-terminal signal peptide (MKYSCCALVLAVLGTELLGSLC^{↓}ST; SEQ ID NO:17) was identified, with the most likely cleavage site located between positions 22 and 23. Based on TMPRED (Stoffel (1993) Biol. Chem. 374:166), there are two other putative membrane-spanning domains in addition to the signal peptide, one spanning amino acids 69-88 and the other spanning amino acids 754-779 near the C terminus. FIG. 3 shows the approximate location of the sulfatase domain within the 871 amino acid HSulf-1 polypeptide.

### Example 3

### Expression Levels of HSulf-1

Northern blot analyses revealed that HSulf-1 encodes a 5.7 kb transcript and a smaller 5.5 kb transcript in a tissue restricted manner. The smaller transcript is an alternatively spliced form of HSulf-1 that is missing exon 20, which shifts the reading frame and codes for a polypeptide that is 790 amino acids in length. These studies showed that expression of HSulf-1 mRNA is higher in small intestine and colon than in ovary. A smaller transcript is present in testis. Spleen, thymus and peripheral blood leukocytes do not express HSulf-1.

To validate the results obtained by SSH, HSulf-1 expression was evaluated in 7 ovarian cancer cell lines and 31 primary ovarian tumors. Northern blotting and semi-quantitative RT-PCR with overlapping HSulf-1 primers spanning the open reading frame demonstrated that HSulf-1 expression was lost in 5 of 7 ovarian cancer cell lines, and was undetectable or markedly diminished relative to normal OSE, the cell of origin, in greater than 80% of the primary ovarian tumors (25/31).

### Example 4

### LOH Analysis of HSulf-1 in Primary Ovarian Tumors

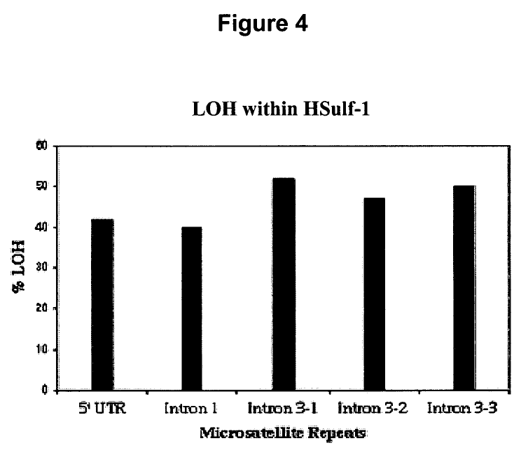

Genomic sequence analysis of HSulf-1 revealed microsatellite markers in the 5′ UTR, one each in introns 1 and 2, and two within intron 3. The primers flanking these repeats are shown in Table 1. Analysis of 30 primary ovarian tumor samples revealed that LOH ranged from 44-53% for these markers (FIG. 4).

TABLE 1
Primers used in LOH analysis in OvCa studies

| | | | | | |
|---|---|---|---|---|---|
| Primer | Sequence | Location | Product | % LOH | SEQ ID |

| | | | | | |
|---|---|---|---|---|---|
| CA1F | 5′-CATCTCCATGTCTGAACTTC | 5′ UTR | 379 bp | 46 (4/9) | 18 |
| CA1R | 5′-ACCTCTTCCTTCAACCTCTG | 26 CA repeats | | | 19 |
| CA2F | 5′-GTCCCTTGTAATGATAATAAG | Intron 1 | 275 bp | 47 (7/15) | 20 |
| CA2R | 5′-GAAGACCAAAGTGGCATC | 70 CA repeats | | | 21 |
| CA3F | 5′-GAGTAAGAAGAGATATTGGAG | Intron 2 | 247 bp | 53 (9/17) | 22 |
| CA3R | 5′-CCTAGCTGTGTGGATCATTGC | 34 CA repeats | | | 23 |
| CA4F | 5′-CGAACTCCTGACCTCAAGTG | Intron 3-1 | 212 bp | 50 (4/7) | 24 |
| CA4R | 5′-CAGAGGGTGGGTGCAGAGTC | 40 CA repeats | | | 25 |
| CA5F | 5′-TAGAATACCTGCACTTCACTG | Intron 3-2 | 193 bp | 50 (10/20) | 26 |
| CA5R | 5′ GAAGACCAAAGTGGCATC | 44 CA repeats | | | 27 |

### Example 5

### HSulf-1 Modulates Drug-Induced Apoptosis

A parental non-expressing primary ovarian carcinoma cell line (SKOV3), vector transfected SKOV3 control, and two HSulf-1 expressing stable clones in SKOV3 (clones 3 and 6) were tested for expression of HSulf-1 using semiquantitative RT-PCR. Only the two HSulf-1 transfectants expressed an HSulf-1 transcript. These clones did not exhibit appreciably different growth properties compared to parental or vector only-transfected cells. The sulfatase activity of clone #6 was measured using 4-MUS as a substrate. There was an approximately 1.7-fold increase in sulfatase activity for HSulf-1 clone 6 compared to vector/parental SKOV3 cell line. Since the 4-MUS substrate used in the sulfatase assay is a non-specific substrate that can be hydrolyzed by most sulfatases, including cellular steroid sulfatases, a higher level of HSulf-1 activity might have been observed if endogenous sulfatase activity was blocked by the steroid sulfatase inhibitor estrone sulfamate (EMATE) or if a substrate specific for HSulf-1 had been used in this analysis.

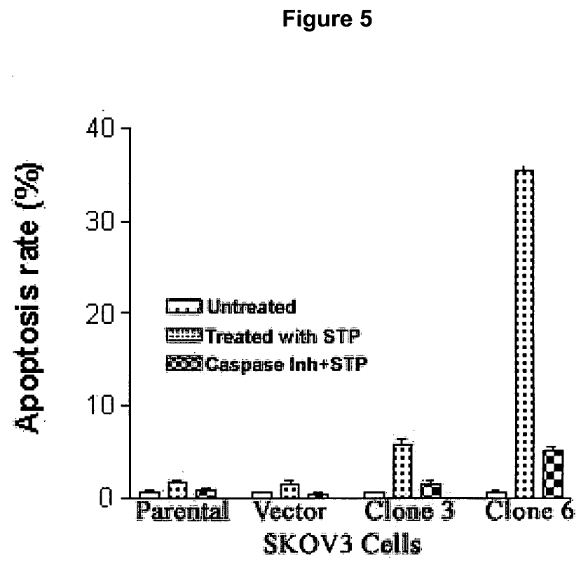

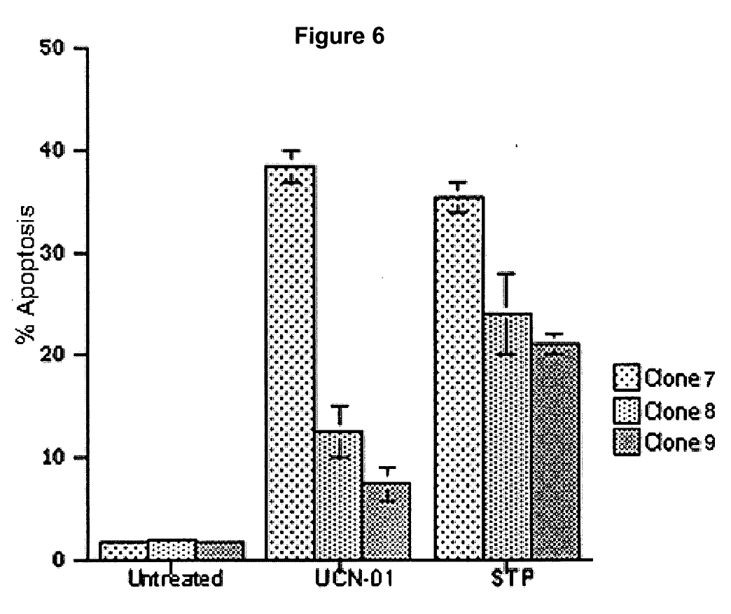

In an effort to further examine the biological consequences of changes in HSulf-1 expression, the transfectants were treated for 5 hours with 1 μM staurosporine, a non-specific kinase inhibitor that broadly induces apoptosis in all cells (Bertrand et al. (1994) Exp. Cell Res. 211:314-321). As shown in FIG. 5, no induction of apoptosis was seen in the parental or vector-transfected SKOV3 cell line after staurosporine treatment. Clones stably transfected with HSulf-1 did not exhibit excess apoptosis in the absence of drug treatment, but instead displayed markedly enhanced caspase-dependent apoptosis when STP was added. Similar results were obtained when the cells were treated with UCN-01 (7-hydroxystaurosporine), a staurosporine analog (Sausville et al. (2001) J. Clin. Oncol. 19:2319-2333; Wang et al. (1996) J. Natl. Cancer Inst. 88:956-965). Examination of DNA fragmentation and cytochrome c release from mitochondria confirmed the results of the morphological assays. In addition, three HSulf-1 expressing stable clones gave similar results upon treatment with STP and/or UCN-01 (FIG. 6). Flow cytometry of these three clones after staining with 7-AAD confirmed the ability of HSulf-1 to modulate staurosporine-induced apoptosis.

In other experiments, stable transfectants were treated for 24 hours with diluent or 5 mM cisplatin. Cells were stained with DAPI and examined for apoptotic morphological changes (nuclear fragmentation) by fluorescence microscopy. Cisplatin induced little apoptosis in parental or vector-transfected cells, but induced apoptosis in 25-40% of HSulf-1-transfected cells. Two aspects of these results deserve emphasis. First, HSulf-1 by itself did not induce apoptosis, but instead modulated the sensitivity of cells to other stimuli. Second, higher expression of HSulf-1 correlated with somewhat higher induction of apoptosis.

Additional experiments demonstrated that HSulf-1 expression also affected proliferation rate. When cells were plated at 100,000 cells per dish and counted at various times, HSulf-1 expressing clones proliferated more slowly than parental or empty vector-transfected clones. The effects of HSulf-1 were not unique to SKOV3 clones. OV207 clones transfected with HSulf-1 also demonstrated increased sensitivity to cisplatin and staurosporine.

### Example 6

### Sulfatase Activity is Required to Modulate Staurosporine-Induced Apoptosis

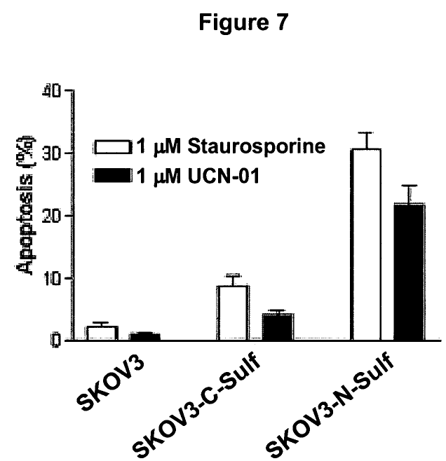

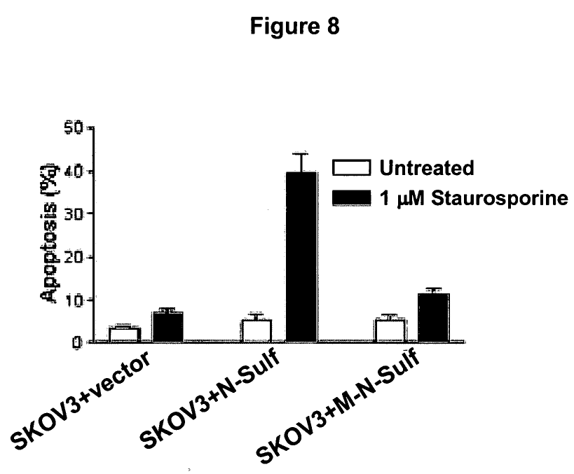

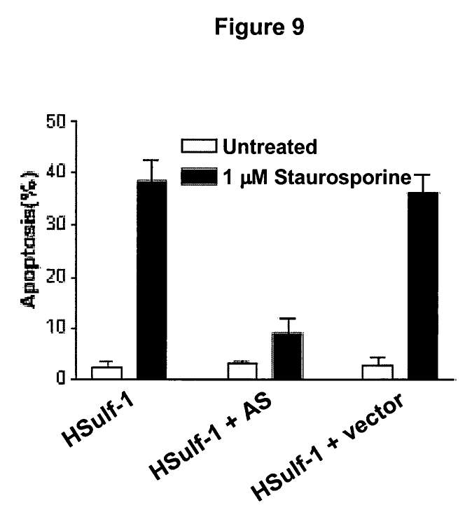

To determine whether an intact sulfatase domain is required for HSulf-1 to modulate staurosporine-induced apoptosis, cells were transiently transfected with an antisense HSulf-1 construct, an expression construct encoding the C-terminal portion or the N-terminal portion of HSulf-1, or an expression construct encoding HSulf-1 having a mutated sulfatase domain. Modulation of apoptosis was more pronounced in cells expressing the N-terminal fragment of HSulf-1, which contains the sulfatase domain, than in cells expressing the C-terminal domain C-Sulf (FIG. 7). Both site directed mutagenesis of the catalytic cysteines C87 and C88 in N-Sulf (mut N-Sulf, FIG. 8) and the presence of an antisense HSulf-1 construct (AS, FIG. 9) attenuated the ability of HSulf-1 to modulate apoptosis, indicating that sulfatase activity was required for this modulation.

### Example 7

### HSulf-1 is Localized to the Cell Surface and is Associated with Decreased Levels of Sulfated HS-GAGs

The avian ortholog of HSulf-1, Qsulf1 was shown to localize to the cell surface through specific interactions of the hydrophilic domain with cell surface components (Dhoot et al. (2001) Science 293:1663-1666). HSulf-1, which has a hydrophilic domain homologous to Q Sulf1, also localized to the plasma membrane, further supporting the possibility that HSulf-1 may modulate growth factor signaling in a manner similar to that observed with Qsulf1. Further analysis demonstrated that tagged HSulf-1 also co-localized with growth factor receptors such as EGFR1 at the cell surface.

To determine whether HSulf-1 expression causes desulfation of cell surface HS-GAGs, cell lines lacking or containing HSulf-1 were stained with an antibody that recognizes native heparan sulfate, including the N-sulfated glucosamine (Clayton et al. (2001) Kidney Int. 59:2084-2094). Parental and vector transfected SKOV3 cells, which do not express HSulf-1, were compared to three different clones expressing full-length HSulf-1. Parental and vector transfected SKOV3 cells showed cell surface staining for N-sulfated glucosamine-containing HS-GAGs, while the cell surface staining was significantly diminished or absent in all three HSulf-1-expressing clones, strongly suggesting that HSulf-1 desulfates HS-GAGs at the cell surface.

### Example 8

### HSulf-1 Modulates Heparin-Binding Growth Factor Signaling

The effect of HSulf-1 on HB-EGF signaling was examined, focusing on events downstream of FGFR occupation. Formation of the FGF2-HSGAG-FGFR ternary complex induces receptor dimerization, activation of the intracellular FGFR tyrosine kinase (Lepique et al. (2000) Endocr. Res. 26:825-832; Selva and Perrimon (2001) Adv. Cancer Res. 83:67-80), receptor autophosphorylation, and binding of the adaptor SNT/FRS, which then activates intracellular signaling pathways including the MAPK pathway (Rapraeger et al. (1991) Science 252:1705-1708). Sustained phosphorylation of p42/ERK1 and 44/ERK2 has been shown to be required for FGF2-induced cell proliferation (Esko (1992) Adv. Exp. Med. Biol. 313:97-106; Rapraeger et al. (1994) Methods Enzymol 245:219-240). To assess the possibility that desulfation of HS-GAGs by HSulf-1 interferes with this signaling, parental, vector-transfected and two HSulf-1 expressing stable clones (7 and 8) were serum starved for 8 hours before the addition of 1 ng/ml of non-heparinated FGF-2 or 100 ng/ml HB-EGF for 15, 30, and 60 minutes. Cells not treated with FGF-2 or HB-EGF served as controls. Blotting with antiphosphoERK1/2 antibody revealed that unstimulated parental or vector-transfected cells had readily detectable constitutive phosphorylation of ERK1 and, to a lower extent, ERK2, whereas clones expressing HSulf-1 had no detectable activation of this pathway. In addition, FGF-2 induced strong, sustained phosphorylation of both ERK1 and ERK2 lasting more than 60 minutes in parental and vector-transfected cells, but only transient and much lower levels of phosphorylation in HSulf-1-expressing clones. Collectively, these results suggest that HSulf-1 not only down-regulates the basal activation of p42/44MAPK, but also inhibits a sustained activation of p42/44MAPK that may be required for cell survival and proliferation. Further analysis confirmed the role of the sulfatase domain in this modulation of MAP kinase activity. Twenty-four hours after transient transfection of SKOV3 cells with vector, wild-type N-Sulf, or a C87, 88A mutant N-Sulf construct, cells were serum starved for 8 hours, treated with 1 ng/ml FGF2 for 10 minutes, and analyzed for MAPK phosphorylation. This analysis revealed that mutation of the active site cysteines abolished the ability of N-Sulf to down-regulate FGF2-induced ERK phosphorylation.

### Example 9

HSulf-1 Modulates Signaling By HB-EGF and Not By Heparin Independent EGF

To determine whether HSulf-1 also modulates other HB-GF signaling, HB-EGF was examined. HB-EGF is postulated to play a role in ovarian carcinogenesis (Gilmour et al. (2001) Cancer Res. 61:2169-2176). Over-expression of EGFR 2 and 4, which mediate the effects of heparin independent EGF and HB-EGF, respectively, has been documented in ovarian cancer cells (Berchuck et al. (1990) Cancer Res. 50:4087-4091; Gilmour et al. (2001) Cancer Res. 61:2169-2176). HB-EGF treatment of vector-transfected cells again resulted in sustained MAPK pathway stimulation, and HSulf-1 transfection diminished this signaling dramatically. To assess whether this attenuation of MAPK signaling reflected the down-regulation of receptor auto-activation, the blots were stripped and probed with phospho-specific anti-EGFR anti-sera that recognize two different autophosphorylation sites, Tyr 1068 and Tyr 992. This analysis demonstrated a marked decrease in EGFR phosphorylation in HSulf-1 clones 7 and 8 compared to vector transfected cells.

In order to show that HSulf-1 modulates only the heparin binding growth factor signaling, serum starved cells were treated with 10 ng/ml EGF for 15 minutes and the levels of phospho-ERK1/2 were measured. Untreated cells served as controls. There was no difference in ERK phosphorylation in HSulf-1 expressing clones 7 and 8 compared to vector transfected control upon EGF treatment, indicating that HSulf-1 modulates signaling by HB-GFs but not by heparin independent growth factors.

### Example 10

### Effects of HSulf-1 Expression on Bcl-2 Family Members

Expression of Bcl-2 family members in control and HSulf-1-transfected SKOV3 cells was evaluated. These experiments were conducted in part because HSulf-1 expression was shown to modulate apoptosis in response to two mechanistically distinct stimuli, cisplatin and staurosporine (see, Examples 5 and 6 herein), suggesting an alteration in the apoptotic machinery rather than in some other process that affects sensitivity in a drug-specific manner (e.g., uptake or metabolism). The experiments revealed, however, that parental, vector-transfected, and HSulf-1-transfected cells exhibited no differences in levels of Bcl-2, Bcl-xL, or Mcl-1 protein. Changes in Bax, phosphorylation of Bcl-2 on Ser⁷⁰, and phosphorylation of Bad on Ser¹¹² likewise failed to account for the altered apoptotic response. Further experiments also showed that there were no differences in the proapoptotic Bcl-2 family member Bak, the X-linked inhibitor of apoptosis protein XIAP, and multiple components of the apoptotic machinery.

### Example 11

### Evaluating HSulf-1 Effects on Growth and Malignant Phenotype

The biological consequences of HSulf-1 loss in ovarian cancer cell lines are examined. If HSulf-1 acts as a tumor suppressor, it should influence the growth of ovarian tumors in culture, as the loss of a tumor suppressor will confer a proliferative advantage. Therefore, experiments are conducted to determine whether engineered over-expression of HSulf-1 results in a decreased rate of proliferation or an enhanced rate of apoptosis. Further studies are conducted to assess whether inactivation of HSulf-1 results in increased proliferation.

Cell growth parameters are assessed using well-established techniques such as cell doubling time, colony formation assays, and growth on soft agar in cell lines with and without HSulf-1 expression. Cell survival parameters are examined by exposing HSulf-1 positive and negative clones to apoptotic stimuli such as serum starvation and treatment with UCN-01 or STP and assessing the apoptotic index. The growth rate of four different vector transfected stable clones is compared with all seven HSulf-1 clones (clones 3 and 6-11) before and after treatment with various concentrations of STP and/or UCN-01 for five hours under serum starved conditions. Antisense expressing clones also are generated in OV202, a cell line with endogenous expression of HSulf-1. These are used in conjunction with parental OV202 and four vector transfected stable OV202 clones in parallel experiments.

To assess soft agar colony forming efficiencies, aliquots containing 0.5×10⁶ tumor cells in 1 mL medium A are plated in gridded 35-mm plates in the medium of Pike and Robinson ((1970) J. Cell. Physiol. 76:8469-8477) containing 0.3% (wt/vol) Bacto agar. After incubation for 14 days at 37° C., colonies containing >50 cells are counted on an inverted phase-contrast microscope.

Two procedures are used to assess chemosensitivities of cancer lines: colony forming assays and direct evaluation of cell death/apoptosis induction. For colony forming assays, subconfluent cells are released with trypsin, plated at a density of 3000 cells/plate in multiple 35-mm dishes containing 2 ml of medium A, and incubated for 14-16 hours at 37° C. to allow cells to attach. Graded concentrations of each drug or equivalent volumes of DMSO (0.1%) are then added to triplicate plates. After a 24 hour treatment, plates are washed twice with serum-free medium and incubated in drug-free medium A for an additional 14 days. Resulting colonies are stained with Coomassie Blue and counted. Diluent-treated control plates typically contain 175-225 colonies. For direct examination of cell death/apoptosis, cells grown in multiple 35 mm tissue culture dishes are incubated in the presence of drug or diluent, harvested at various time points and processed for cell viability and apoptosis studies. The percentage of cells that are actively undergoing apoptosis is quantitatively determined using flow cytometry and the Annexin V-PE kit from BD Pharmingen, following the manufacturer's protocol.

### Example 12

### Determining the Effect of Loss of HSulf-1 on Malignant Phenotype

Studies are conducted to determine whether ovarian cancer xenografts progress more rapidly in mice with HSulf-1 negative cells than in HSulf-1 positive cells, and whether forced expression of HSulf-1 decreases formation of tumors in mice exposed to UCN-01/STP. Four to five-week old nude mice (10 in each of four groups) are injected with 2×10⁷ cells/100 ml each subcutaneously in the hind flanks with two different clonal vector transfected and HSulf-1 expressing cell lines. The tumorigenic potential of vector transfected lines is assessed and compared to that of HSulf-1 expressing stable lines. For each stable cell line clone to be tested, 10 mice are necessary. This sample size per condition allows 80% power to determine a 50% change in oncogenic potential that is statistically (and biologically) significant (alpha=0.05). Mice are monitored and growth of subcutaneous tumors are measured in two dimensions daily for 20 weeks, with the tumor volume calculated according to the formula V=a²b/2, where a is the shortest and b the longest diameter. Data are analyzed using repeated measurements ANOVA (analysis of variance) including a growth curve analysis. Animals are observed for up to 20 weeks for measurement of latency time or the failure to develop tumor nodules at the implantation site. At the time of sacrifice, tumors are removed for histological assessment and storage in liquid nitrogen for subsequent studies to ensure that the HSulf-1 expression is still retained in these samples.

Treatments are initiated when tumors reach an average diameter of 4 mm. Mean volumes at 28 days post exposure are compared using a two-tailed, two-sample t-test of log transformed tumor volumes between each pair of treatments. Tumor response is studied using tumor growth and growth delay time assays. At the time tumors reach 4-5 mm in diameter, 10 of the 15 animals in each group are treated with a cytotoxic agent, whereas the other 5 are untreated controls. Treatments: UCN-01 is given continuously for 7 days using an Alzet osmotic pump (4.0 g/L/h or approximately 3.2 mg/kg/day). After completion of treatment, animals are examined two or three times each week. The volumes of palpable tumors are calculated, and the growth rate of each individual tumor is plotted. If frank regressions are not observed, data are expressed as the delay in time required for the tumors to reach a mean volume of 1 cm³. Measurable subcutaneous SKOV-3 tumors have been reported in 3-4 weeks using nude mice, but it may be preferable to implant the first pump after 1-2 weeks (when the tumors reach a size requiring neovascularization). Pilot studies are performed to validate the timing of pump implantation and tumor measurement before the critical experiments.

### Example 13

### Identifying Natural Substrates for HSulf-1

Prior to the experiments with purified HSulf-1 enzyme, the optimal pH for the enzyme is determined using buffers ranging in pH from 4.0 to 10.0, with 4-MUS as the substrate. The calculated pI of full-length HSulf-1 is 9.23, consistent with a non-lysosomal location for its function. Reactions of HSulf-1 enzyme are performed with disaccharides derived from heparan sulfate that are mono-, di-, or tri-sulfated. Substrates include DUA-2S-[1AE4]-GlcN (Ddi-mono2S), DUA-2S-[1AE4]-GlcNAc (aDdi-mono2S), DUA-[1AE4]-GlcN-6S (Ddi-mono6S), DUA-[1AE4]-GlcNAc-6S (aDdi-mono6S), DUA-[1AE4]-GlcNS (Ddi-monoNS), DUA-2S-[1AE4]-GlcN-6S (Ddi-di(2,6)S), DUA-2S-[1AE]-GlcNAc-6S (aDdi-di(2,6)S), DUA-2S-[1AE4]-GlcNS (Ddi-di(2,N)S), DUA-[1AE4]-GlcNS-6S (Ddi-di(6,N)S), and DUA-2S-[1AE4]-GlcNS-6S (Ddi-tri(2,6,N)S). The DU residues are removed with either glycuronidase or mercuric acetate to produce monosaccharide substrates without DU. Unsaturated tetrasaccharides also are tested as possible substrates for HSulf-1, as are chemically modified heparins (e.g., Neoparin). Disregarding the variation in the two uronic acid epimers iduronic acid and glucuronic acid, these 10 disaccharides are all the sulfated disaccharides known to be derived from heparan sulfate. Each of these disaccharides, and the two non-sulfated disaccharides DUA-[1AE]-GlcN (Ddi-nonS) and DUA-[1AE4]-GlcNAc (aDdi-nonS), are separated by high performance capillary electrophoresis (CE) using a 50 μM (inner diameter), 375 μM (outer diameter) and 62 cm long fused silica capillary (ISCO). The CE system is operated in reverse polarity mode by applying the sample at the cathode and running with 20 mM H₃PO₄ adjusted to pH 3.5 with 1 M Na₂HPO₄. The capillary is washed before use with 0.5 ml of 0.5 M NaOH, followed by 0.5 ml of distilled water and then 0.5 ml running buffer. Samples are applied using vacuum injection, and electrophoresis is conducted at 20 kV with detection at 232 nm. Each of the sulfated disaccharides is incubated in a reaction with active HSulf-1 enzyme at the optimal pH for HSulf-1 activity. After incubation at 25° C. or 37° C. for varying time periods, sulfatase activity is assessed from the disappearance of sulfated substrates and the appearance of less sulfated or unsulfated substrates by CE. Control reactions are performed with extracts from Sf9 or “High Five” cells not transfected with HSulf-1 bacmids to control for potential sulfatase contamination in the extracts. Sulfated tetra-, hexa-, or octasaccharides also can be used for additional experiments.

In a second set of experiments to test the hypothesis that HSulf-1 desulfates cell surface HS-GAGs, the effect of HSulf-1 expression on the composition of cell surface HS-GAGs purified from the HSulf-1-negative cell line SKOV3 is examined and compared to the vector-transfected stable cell line SKOV3-Vector, the two HSulf-1 transfected stable clones 7 and 8, and the high HSulf-1 expressing cell lines OV167 and OV202. HS-GAG fragments are collected by incubating 90-100% confluent cells with 1.5 ml of PBS containing a mixture of the heparin- and heparan sulfate lyases (heparin lyases I, II and III, Seikagaku) at 37° C. on a shaker for 1 hour. The supernatant is pooled into a tube, centrifuged for 8 minutes at 4500×g, boiled for 15 minutes, and filtered. HS-GAG fragments are bound to an Ultrafree-DEAE membrane that has been equilibrated with sodium phosphate, pH 6.0 with 0.15 M NaCl. The fragments are washed with the same buffer and eluted with 0.1 M sodium phosphate buffer pH 6.0 with 1.0 M NaCl. The fragments are then concentrated and buffer-exchanged into ultra-pure water by application to a Microcon filter (MWCO=3000 Da). The samples are digested overnight with a mixture of heparin lyases I, II, and III, 1 milliunit each) in 25 mM sodium acetate and 1 mM calcium acetate, pH 7.0. Following incubation with the combined heparin lyases, the glycosaminoglycan chains are degraded almost completely (>90%) to delta-disaccharides, which are then separated by CE as described above. The identity of the disaccharide peaks is determined by comigration with known standards. This method yields a compositional analysis profile of all the sulfated and unsulfated disaccharide components of the HS-GAGs from each cell line, and allows analysis of the differences in sulfation states of HS-GAGs from HSulf-1-negative versus HSulf-1-expressing cell lines.

Several heparin-binding proteins interact with heparin/HS-GAGs through consensus heparin/heparin sulfate binding motifs XBBBXXBX (SEQ ID NO:28) and XBBXBX (SEQ ID NO:29), thought to be important for ionic interactions with glycosaminoglycan ligands (Cardin and Weintraub (1989) Arteriosclerosis 9:21-32; and Hileman et al. (1998) Bioessays 20:156-167). Human HSulf-1 contains several highly basic peptide stretches; one of these at amino acid positions 402-407 (NKKAKI; SEQ ID NO:30) conforms to the binding motif pattern XBBXBX (SEQ ID NO:29). Other highly basic peptides LRKKEESSK (420-428; SEQ ID NO:31), LKRRKP (668-673; SEQ ID NO:32), VKKQEKLK (690-697; SEQ ID NO:33), and RRRKKERKEKRRQRKG (723-738; SEQ ID NO:34) also are present in HSulf-1 protein. Basic residues at amino acid positions 403 and 404 are altered to glutamine by site directed mutagenesis, and experiments are conducted to determine whether this alteration alters the phosphorylation levels of ERK1/2 compared to wild-type sequence.

### Example 14

### Tumor-Specific Mutations of HSulf-1

The loss of expression of a gene could also be due to tumor specific mutations that may alter an amino acid or the presence of nonsense mutations that results in a truncated protein. Either of these events could lead to a functional inactivation of a gene such as HSulf-1. To determine if there are any inactivating mutations in HSulf-1 at the genomic level, all 23 exons of HSulf-1 are amplified using template nucleic acids obtained from a panel of 100 primary tumors and from seven cell lines (3 and 6-11) described herein, and evaluated using denaturing high pressure liquid chromatography (DHPLC) analysis.

DHPLC is a novel automated separation technology that compares two or more chromosomes as a mixture of denatured and reannealed PCR amplicons. Under partially denaturing conditions, heteroduplexes generally have shorter retention times than homoduplexes. By employing a novel nonporous stationary phase, DNA fragments up to 1 kb can be analyzed within a few minutes using on-line UV detection. PCR products without any additional treatment are subjected to a 3 minutes 95° C. denaturing step followed by gradual reannealing from 95-65° C. over a period of 30 minutes. The samples are then applied to the DHPLC column and eluted with a linear acetonitrile gradient of 0.45% per minute at a flow-rate of 0.9 ml/min. The start- and end-points of the gradient are adjusted according to the size of the amplicon. The predicted temperature required for successful resolution of heteroduplexes from homoduplexes can be obtained from the DHPLC melt program (World Wide Web at “lotka” dot “stanford” dot “edu” slash “dhplc” slash “meltdoc” dot “html”). Amplicons that appear to detect heteroduplexes by DHPLC analysis are cleaned with exonuclease and shrimp alkaline phosphatase as described (US Biochemical), and sequenced with fluorescent terminators on an ABI Prism 377 Sequencer (Perkin Elmer). However, for the size-dependent separation of two DNA fragments, DHPLC is performed at 45° C., a non-denaturing HPLC condition (nDHPLC). To prepare DNA templates for PCR amplifications, DNA from ovarian tumors and from normal ovarian epithelium are isolated and diluted to a final concentration of 25 ng/ml. DHPLC detects sequence mismatches based on the separation of heteroduplexes from homoduplexes. In general, a heteroduplex indicates the presence of a mutation or a polymorphism and a homoduplex, a wild-type sequence. If the DHPLC analysis shows a heteroduplex profile for a certain sample, only the two DNA samples in the mixture need to be sequenced to determine if a mutation is present.

Methylation is analyzed as an alternative mechanism for inactivating the transcription of HSulf-1. Human cancers can have aberrant methylation (e.g., hypomethylation or hypermethylation) of DNA, which may lead to increased chromosomal instability in cancer cells. Another form of aberrant methylation involves region specific hypermethylation of CpG islands in the promoter sequences of specific genes (Baylin et al. (1998) Adv. Cancer Res. 72:141-196; and Myohanen et al. (1998) Cancer Res. 58:591-593). Hypermethylation of a promoter region can result in transcriptional inactivation (Costello et al. (2000) Brain Tumor Pathol. 17:49-56; Esteller et al. (2000) J. Natl. Cancer Inst. 92:564-569; and Jarrard et al. (1998) Cancer Res. 58:5310-5314).

The 5′ promoter and the introns of HSulf-1 do not appear to contain any “canonical” CpG islands. However, differential methylation is not limited to CpG islands within the promoter or an intron (Shridhar et al. (2001) Cancer Res. 61:4258-4265). Methylation specific PCR (MS-PCR) is used to look for other potentially “methylatable” sequences in the 5′ end of HSulf-1 to determine if any one of these sequences shows differential methylation in tumors that show loss of expression of HSulf-1 compared to tumors that express HSulf-1.

For MS-PCR, DNA is modified with sodium bisulfite according to Herman et al. (1996) Proc. Natl. Acad. Sci. USA 93:9821-9826) with the following modifications: 1-1.5 mg of DNA is digested with EcoRI in a 50 ml reaction overnight. The digested DNA is extracted once with phenol-chloroform-isoamyl alcohol (25:24:1) and precipitated with 1/10 volume of 5.0 M ammonium acetate and 100% ethanol in the presence of 1 ml of 20 mg/ml glycogen (Boehringer Mannheim, Indianapolis, Ind.). The DNA pellet is washed twice with 70% ethanol and the DNA is taken up in 90 ml of 10 mM Tris (pH 7.5) plus 1 mM EDTA (TE). Ten ml of freshly prepared 3.0 M NaOH is added to each sample and the DNA is denatured at 42° C. for 30 minutes. After the addition of 10 μl of distilled water, 1020 μl of 3.0 M sodium bisulfite (pH 5.0) and 60 μl of 10 mM hydroquinone, the samples are incubated in the dark at 55° C. overnight (16-20 hours). Modified DNA is purified using the Wizard purification system (Promega) according to the manufacturer's instructions, followed by denaturation with 0.3 M NaOH for 15 minutes at 37° C. The DNA is eluted in 50-100 ml of TE and stored at −20° C. in the dark. Samples are sequenced to determine their methylation status. Methylated Cs are resistant to bisulfite modification, whereas unmethylated Cs are converted to Ts. Therefore, methylated Cs are read as Gs, and unmethylated Cs (converted to Ts) are read as As in the complementary strand.

### Example 15

### Identification of Mediators of HSulf-1-Induced Changes in Cellular Behavior

Two sets of cloned isogenic cell lines are available: SKOV3 transfected with vector or HSulf-1, and OV207 cells transfected with the same two plasmids. In initial experiments, total cellular RNA is isolated from SKOV3 vector-transfected and SKOV3 clone 7. The RNA is analyzed for degradation using an Agilent Bioanalyzer 2100 (Agilent Technologies, Palo Alto, Calif.). If the 28/18 S rRNAs are in the correct ratio and there is no noticeable degradation appearing in the 4-5S range, procedures including reverse transcription, in vitro translation, and biotin labeling of the resulting cRNA are conducted. The biotinylated cRNA is degraded to a uniform size, as determined using the Agilent Bioanalyzer, to permit more rapid hybridization. A test hybridization is then performed on a Test3 chip, which contains a small number of genes and permits determination that the background hybridization is proper, correct sites are labeled, and intensity is adequate. Once these quality control checks are performed, the cRNA is hybridized to Affymetrix HG-U133 microarrays (Affymetrix, Inc., Santa Clara, Calif.), which consist of two GENECHIPS® containing almost 45,000 probe sets representing more than 39,000 transcripts derived from ˜33,000 well-substantiated human genes. Bound cRNA is detected using phycoerythrin-conjugated streptavidin and quantitated on an Affymetrix GENEARRAY® Scanner. The output consists of fluorescence intensities representing hybridization to each of the 406,000 unique oligos on these arrays. Affymetrix software is utilized to examine internal controls (multiple matched and mismatched oligos for each transcript) and arrive at an average hybridization intensity for each represented transcript. If one or more biochemically plausible changes in expression of possible regulators of drug-induced apoptosis is identified, the potential involvement of this change in altered sensitivity to cisplatin-induced apoptosis is verified by altering expression of the transcript of interest.

### Example 16

### Effect of HSulf-1 Down Regulation on the Biology of Ovarian Surface Epithelial Cells

Using cationic lipid-mediated transfection, OSE(tsT) cells grown at 34° C. are transfected with pcDNA3.1 containing HSulf-1 cDNA in the antisense orientation under the control of the strong constitutive cytomegalovirus promoter (or empty vector as a control). Forty-eight hours after transfection, G418 is added at a concentration of 800 μg/ml to select for stable transfectants. Once colonies form, individual clones are isolated using cloning rings and handled as separate lines.

One of several assays is utilized to screen for clones with HSulf-1 down regulation. If anti-HSulf-1 antibodies are available, clones are screened by immunoblotting or immunohistochemistry. If antibodies are not available, clones showing enhanced HSPG sulfation upon staining with sulfation-sensitive 10E4 antibody are studied. Because HSulf-1 appears to contribute ˜50% of total sulfatase activity measured by 4-methylumbelliferyl sulfate, down regulation of HSulf-1 also can be confirmed using a sulfatase assay such as that described in Example 1.

Once clones with diminished HSulf-1 are identified, their biological properties are explored by comparison to vector-transfected clones. Proliferation rates are assessed by plating 10⁵ cells in replicate 100 mm plates in medium A (1:1 MCDB 105:Medium 199 supplemented with 15% fetal bovine serum) at 34° C. and examining cell number at daily intervals. The requirement for active T antigen for continued proliferation is assessed by plating 10⁵ cells in replicate 100 mm plates in their regular medium at 39° C. and examining cell number at daily intervals. Requirement for adhesion for continued proliferation is assessed by plating 10⁴ cells in 0.3% agar (over a layer of 0.5% agar) in replicate gridded 35 mm plates and examining their ability to form colonies at 34° C. Ability to survive in the absence of cytokines (resistance to growth factor-induced apoptosis) is assessed by plating 10⁶ cells in replicate 100 mm plates in medium A lacking serum and examining cell number and viability at daily intervals. Resistance to detachment-induced apoptosis is assessed by incubating trypsinized cells in sterile test tubes containing medium A at 34° C. and examining cell number and viability at daily intervals. Resistance to cisplatin-induced apoptosis is assessed by treating 10⁶ cells in replicate 100 mm plates with varying cisplatin concentrations (from 1-30 μM) for 24 hours and staining with DAPI before morphological assessment of apoptotic changes.

If HSulf-1 down regulation results in growth in soft agar (an in vitro surrogate for malignant transformation), the effect of HSulf-1 down regulation on tumor formation in nude mice is evaluated. In brief, clones transfected with vector or HSulf-1 antisense cDNA are assayed by implanting 10⁷ cells in 0.25 ml phosphate-buffered saline into the flanks of 10 CD-1/NU athymic mice. As a positive control, 10⁷ SKOV3 cells from ATCC, which are known to form tumors in nude mice, are injected into mice. Animals are observed for 60 days for tumor formation; and any resulting tumors are examined to confirm that they are of ovarian origin and establish their histological subtype.

### Example 17

### Assessment of HSulf-1 Effects on Sensitivity to Drugs in Vitro

For these studies, clones that differ only in HSulf-1 expression (e.g., parental SKOV3, vector-transfected cells, and clones 7-9) are utilized. In a series of separate experiments, aliquots of each culture are treated with diluent or varying concentrations of paclitaxel (10-100 nM), topotecan (20-400 nM), gemcitabine (5-100 nM), or doxorubicin (10-400 nM). At 24, 48 and, if necessary, 72 hours after the start of drug treatment, adherent cells are harvested by trypsinization. Since apoptotic changes typically are found almost exclusively in cells that detach from tissue culture plates, nonadherent cells are saved and quantitated separately or added back to the trypsinized adherent cells. After sedimentation, cells are fixed in 3:1 methanol:acetic acid, stained with Hoechst 33258 or DAPI, and examined by fluorescence microscopy for apoptotic morphological changes. Data are expressed as the percentage of total cells that are apoptotic after each treatment.

Because cells expressing HSulf-1 might undergo apoptosis faster but the same number of cells might ultimately be killed by drug treatment, the effects of HSulf-1 on long-term survival are examined using colony forming assays. In brief, replicate aliquots of each clone are plated in triplicate 35 mm dishes. After an overnight incubation to allow cells to adhere, triplicate sets of plates are treated for 24 hours with increasing concentrations of cisplatin, paclitaxel, topotecan, gemcitabine, or doxorubicin. At the completion of the incubation, cells are washed twice and incubated in drug-free medium until colonies form. After colonies are counted under low power magnification, data are expressed as a ratio of the number of colonies in plates treated with each drug concentration to number of colonies in plates treated with diluent. In this way the ability of HSulf-1 to modulate effects of the agents on proliferative potential (“clonogenic survival”) is determined. If, however, the colony forming assays yield results that fail to agree with the apoptosis assays, long-term survival also is examined using outgrowth assays. In particular, the ability of vector- vs. HSulf-1-transfected clones to repopulate the flasks is evaluated after a drug dose that kills several logs of cells. These types of repopulation studies appear to correlate with the effects of genetic changes on drug sensitivity in vivo.

As described in Example 5, HSulf-1 re-expression sensitized SKOV3 cells to staurosporine and cisplatin. If these studies show that HSulf-1-expressing cells are selectively sensitized to cisplatin but not to other agents such as paclitaxel, topotecan, gemcitabine and doxorubicin, the mechanism of selective sensitization is examined. Paired cell lines are assayed for cisplatin accumulation as well as formation and removal of platinum-DNA adducts. Depending upon the results of these assays, known mechanisms of cisplatin uptake and detoxification are examined.

If HSulf-1 re-expression sensitizes ovarian cancer cells to some agents but not others, the sensitization pattern is confirmed using OV207 clones 1 and 4. Follow up experiments then focus on the question of how HSulf-1 expression differentially affects some drugs but not others.

### Example 18

### Effects of HSulf-1 on Drug Sensitivity In Vivo

In vivo studies are conducted to determine whether the difference in drug sensitivity conferred by HSulf-1 re-expression is unique to the flank xenograft model or is also seen when ovarian carcinoma cells grow orthotopically. After a pilot study to confirm that the cell lines form tumors in nude mice, clone 7 (high HSulf-1 expression) and empty vector transfected (undetectable HSulf-1) SKOV3 derivatives are compared in vivo. Parental SKOV3 cells from ATCC are known to form xenografts in the flanks of CD-1/NU mice (Hirasawa et al. (2002) Cancer Res. 62:1696-1701). Clone 7 cells (1×10⁷ cells) in 0.25 ml phosphate-buffered saline are injected subcutaneously into the right flank of CD-1/NU athymic mice, and 1×10⁷ empty vector-transfected cells are injected subcutaneously into the left flank. Once the tumors reach an average diameter of 5-7 mm (possibly about 7 days after injection), animals are randomly assigned to five groups that are treated as follows:
Group 1: untreated control
Group 2: cisplatin 4 mg/kg IP on days 1, 5 and 9, where day 1=first day of drug injection
Group 3: paclitaxel 25 mg/kg IP on days 1, 5 and 9
Group 4: topotecan 0.625 mg/kg IP on days 1-20
Group 5: gemcitabine 240 mg/kg IP on days 1 and 8
   Another group also may be treated with liposomal doxorubicin. Importantly, these studies are performed only with agents whose activity is modulated by HSulf-1 in the experiments disclosed in Example 17. While it is clear that the cytotoxicity of cisplatin is affected by HSulf-1, other agents are included in this experiment only if their activity is modulated in vitro.

Agents are administered intraperitoneally. Paclitaxel is administered using a clinical formulation containing ethanol and polyethoxylated castor oil. Cisplatin and gemcitabine are administered in PBS. Topotecan is administered in 0.85% NaCl, pH 5.5. For each animal, the bidirectional diameters of the tumors are measured twice a week with calipers, and tumor volumes are calculated. Animals also are weighed twice weekly to assess toxicity, and are sacrificed at any time they appear to experience discomfort or at the time tumors reach 1.5 cm in diameter.

The untreated group includes three extra animals that are sacrificed when the tumors reach 8 mm in diameter so that tissue can be harvested and examined for HSulf-1 expression. Small aliquots of tumors are embedded in OTC medium in preparation for frozen sections, which are subjected to immunohistochemical staining when anti-HSulf-1 antibodies become available. The bulk of each tumor is snap frozen for subsequent RT-PCR analysis. After RNA is purified and reverse transcribed as described below, HSulf-1 message is quantitated by Light Cycler analysis.

If tumors derived from vector-transfected cells grow more rapidly than those derived from clone 7 or 9, resulting in a disparity in the time needed for the tumors to triple in volume even in the control animals, the proposed statistical analysis is altered to take into account the difference in growth rate in the absence of drug treatment. For example, the ratio of the time required for the HSulf-1-transfected vs. vector-transfected xenografts to triple in size in each animal is calculated. After calculating the means and standard deviations of this ratio for each group, the ratios between groups are compared. If a particular agent, e.g., cisplatin, is more effective in HSulf-1-transfected cells than in vector transfected cells, this ratio should be significantly larger (i.e., the growth delay in the HSulf-1 transfected cells should be preferentially increased) for that drug. All analyses are performed as described above except that ratios would be substituted for the tripling times of individual tumors.

The results of the preceding experiments are followed up using an orthotopic model combined with systemic drug administration. If vector-transfected and HSulf-1-transfected SKOV3.ip1 clones are produced, aliquots containing 2-10×10⁶ log phase cells in 0.5 ml PBS are injected intraperitoneally into CD-1/NU mice. Pilot experiments are performed to confirm that both cell lines produce intraperitoneal carcinomatosis. In subsequent studies, mice are injected with control or HSulf-1-transfected cells and randomized 5 days later to receive saline or drug. Experiments are initially on a drug that is shown in the experiments of Example 17 to be affected by HSulf-1 expression in vitro. For example, topotecan can be administered by gastric gavage, and gemcitabine can be administered intravenously.

### Example 19

### Decreased HSulf-1 Expression and Drug Sensitivity in Patient Subsets with Good vs. Poor Outcomes

HSulf-1 expression and drug sensitivity are examined in a set of patients previously treated for stage III ovarian cancer (serous, endometrioid or mixed serous/endometrioid), and whose clinical responses represent the two ends of the spectrum. All patients were treated with a platinum-containing regimen, with the vast majority receiving platinum+paclitaxel. At one end of the spectrum are patients in the good outcome group, with a median time to recurrence (time from surgery to start of second-line treatment) of 35.5 months. At the other end of the spectrum is the poor outcome cohort, with a median time to recurrence of 8.7 months.

HSulf-1 mRNA levels are determined using quantitative PCR to evaluate whether down regulation is more common in one group than the other. Total RNA is extracted from tissue blocks obtained from all patients at the time of initial diagnostic surgery, using the RNAeasy mini kit (Qiagen). cDNA synthesis is performed using a SUPERSCRIPT™ II RNase H-reverse transcriptase kit (Invitrogen/Life Technologies) to transcribe 1-5 μg of total RNA with 1 μg of 500 μg/ml oligo(dT)12-18 primer. Light Cycler RT-PCR is then performed. In brief, 50-100 ng of reverse transcribed cDNA is mixed with the primers F1 (5′-AATGCTGCCCATCCACATG-3′; SEQ ID NO:35) and R1 (5′-CAGAATCATCCACTGACATCAAAGT-3′; SEQ ID NO:36) plus RPS9-F (5′-TCGCAAAACTTATGTGACCC-3′; SEQ ID NO:37) and RPS-R (5′-TCCAGCACCCCCAATC-3′; SEQ ID NO:38). Duplex PCR amplification is carried out with a Light-Cycler (Roche) in the presence of SYBR-Green dye using 1 minute at 95° C. for initial denaturation and 40 cycles at 95° C. (10 seconds), 58° C. (15 seconds), and 72° C. (20 seconds) for amplification, with measurement of fluorescence at the end of each cycle. After the 40th cycle, melting curve analyses are performed with Light-Cycler software by denaturing the sample at 95° C., rapidly cooling down to 65° C. for 15 s, and measuring the fluorescence as the sample temperature is gradually raised to 95° C. at 0.1° C./sec. Each run includes a negative control as well as multiple aliquots of a positive control to confirm the linear relationship between copy number and cycle number. This positive control is prepared as follows: cDNA is prepared from OV202 cells that express HSulf-1 using an oligo-dT primer and MLV reverse transcriptase. Using the forward primer 5′-CCACCTACCACTGTCCGAGT-3′ (SEQ ID NO:39; Tm=60° C.) and the reverse primer 5′-TCTGCCGTCTCTTCTCCTTC-3′ (SEQ ID NO:40; Tm=60° C.), cDNA is synthesized (product size 379 bp). After electrophoresis in a 1% low melting temperature agarose gel, a band of the expected size is excised and eluted into Tris-HCl using a DNA elution kit (Qiagen). After the eluted DNA is quantitated by absorbance at 260 nm and sequenced, standards are prepared at concentrations of 109, 108, 107, 106, 105, 104, and 103 copies/ml. These standards are included in each quantitative PCR run and used to calculate the copy number in the experimental sample. The result is expressed as a relative ratio of product (copies/ml) to the housekeeping gene GAPDH (copies/ml) from the same RNA (respective cDNA) samples.

Results of this analysis provide quantitative data expressed as a ratio of HSulf-1 transcripts/GAPDH transcript for 81 stage III tumors from 32 good outcome and 49 poor outcome patients. These values are compared to the mean of multiple pooled normal ovarian epithelial cell brushings. A cancer specimen is considered to have diminished HSulf-1 expression if the HSulf-1/GAPDH transcript ratio is <20% of the mean ratio in the pooled normal samples. Fisher's exact test is performed to test the null hypothesis that the proportion of samples with diminished expression is equal in the two experimental groups. Preliminary estimates indicate that the frequency of HSulf-1 down regulation is ˜70% (18/26) in serous and endometrioid tumors as a whole. If the incidence of HSulf-1 down regulation is 85% in the poor outcome group, power calculations indicate that this sample size has 88% power to detect a decrease in frequency of HSulf-1 down regulation to 50% at alpha=0.05 in the good outcome group. If the frequency of HSulf-1 down regulation is lower in the poor outcome group, the power to detect a decreased frequency in the good outcome group is correspondingly diminished.

If anti-HSulf-1 antibodies are available, the relationship between HSulf-1 expression and clinical outcome is examined using this alternative approach. A major advantage is the availability of archival material from larger numbers of patients with outcomes at both ends of the spectrum. In brief, 4 mm thick sections of formalin-fixed, paraffin-embedded material are deposited on slides. Samples are deparaffinized for 30 minutes in xylene, rehydrated (3 washes each) in 100%, 90% and 80% ethanol, and incubated for 5 minutes at room temperature in 3% H₂O₂ in methanol to inactivate any endogenous peroxidases. Microwave-induced antigen retrieval is performed by immersing slides in 0.01 M citric acid (pH 6.0) and heating for 30 minutes. Samples are washed in calcium-free, magnesium-free phosphate-buffered saline (PBS), incubated in 1% bovine serum albumin (BSA) in PBS to block nonspecific binding sites, reacted for 1 hour at 37° C. with primary antibodies diluted in PBS/BSA, washed 6 times with PBS over is 20 minutes, incubated for 30 minutes at room temperature with biotinylated secondary antibody (1:150, Zymed, San Francisco, Calif.) in PBS/BSA, washed 6 times with PBS over 20 minutes, incubated with horseradish peroxidase-labeled streptavidin (1:500, Zymed) in PBS/BSA, washed 6 times over 20 minutes, and stained with aminoethylcarbozole and 0.02% H₂O₂. For this assay, SKOV3 and OV207 cells (lacking HSulf-1), and normal OSE are included in each batch of slides as negative and positive controls, respectively. Staining is graded as 0 (no reactivity), 1+ (weakly reactive), 2+ (moderate reactivity), 3+ (strongly positive). To explore the relationship of the staining with clinical outcome, the four possible outcomes for staining (0, 1+, 2+, and 3+) are dichotomized into two groups, high vs. low or negative (0) vs. positive (1+, 2+, 3+). Fisher's Exact test is used to detect any significant relationships between these dichotomous variables and treatment response. With a sample size of 100 pts, 40% with >30 month disease-free survival and 60% with shorter disease-free survival, there is 87% power to detect a difference in incidence of HSulf-1 down regulation of 85% in the poor outcome group vs. 55% in the good outcome group.

### Example 20

### HSulf-1 Expression and Disease Status at Second Look Laparotomy (SLL)

One hundred patients are randomly selected who have been diagnosed with stage III serous, endometrioid, or mixed serous/endometrioid ovarian cancer and who have received paclitaxel/platinum chemotherapy between initial diagnostic surgery and SLL. Once specimens from initial diagnostic surgery of these patients are provided, Light Cycler RT-PCR or immunohistochemistry is performed as described above. Each sample is scored as showing normal or diminished HSulf-1 expression.

Patients are divided into those with and those without detectable disease at SLL. The frequency of HSulf-1 down regulation in these two groups is examined using the strategies described above. If the incidence of HSulf-1 down regulation in patients with positive SLL is 0.85, there is 87% power to detect a decrease in incidence of HSulf-1 down regulation to 0.55 in the negative SLL (good outcome) group at the alpha=0.05 level. If the incidence of HSulf-1 down regulation in patients with positive SLL is only 0.70, the power to detect a significant decrease in the incidence of HSulf-1 down regulation in the patients with negative SLL is somewhat lower.

### Example 21

### Determination Whether Effects of HSulf-1 Down Regulation are Reversible

These studies are very similar to those described in Examples 17 and 18 except that signal transduction inhibitors are substituted for anticancer drugs. In brief, multiple clones that differ only in HSulf-1 expression are treated with diluent or varying concentrations of Iressa (10-800 nM), BAY 37-9751 (5-50 μM), CI-1040 (1-10 μM ), or 1L-6-hydroxy-methyl-chiro-inositol 2-(R)-2-O-methyl-3-O-octadecylcarbonate (1-25 μM ). Half of each aliquot of cells is fixed, stained with Hoechst 33258, and examined for apoptotic morphological changes. Data are displayed graphically (% apoptosis vs. drug concentration for each cell line), and sensitivity of various cell lines is compared. The remaining cells are solubilized for SDS-PAGE followed by immunoblotting so that aliquots containing equal protein can be probed with antisera recognizing tyrosine phosphorylated EGFR, phosphorylated Erk1 and Erk2, or phospho-p70^{S6} kinase (a downstream target of Akt) to assess the efficacy of Iressa, BAY 37-9751 and CI-1040, or 1L-6-hydroxymethyl-chiro-inositol 2-(R)-2-O-methyl-3-O-octadecylcarbonate, respectively. Blots are stripped and reprobed with antibodies to EGFR, Erk1/2 or total Akt to confirm equal loading.

### Example 22

### Effects of Signal Transduction Inhibitors in Combination with Cisplatin, Paclitaxel, Topotecan or Gemcitabine

If vector-transfected and HSulf-1-transfected cells are equally sensitive to Iressa, BAY 37-9751, CI-1040, and 1L-6-hydroxymethyl-chiro-inositol 2-(R)-2-O-methyl-3-O-octadecylcarbonate, the effects of these agents on sensitivity to conventional drugs utilized to treat ovarian cancer are examined. Previous studies have demonstrated that Iressa sensitizes cancer cells to cisplatin, paclitaxel and topotecan in vitro. Testing of combinations in vivo has been more limited, although Iressa is known to enhance response of xenografts to cisplatin or paclitaxel. The MEK1/2 inhibitor PD98059 likewise sensitizes a variety of cell lines, including ovarian cancer cell lines, to cisplatin and paclitaxel in vitro.

The effect of combining the signal transduction inhibitors with chemotherapeutic agents is evaluated in vitro using the induction of apoptosis as an endpoint. Each signal transduction inhibitor is tested in turn with chemotherapeutic drugs. For example, HSulf-1-deficient cells (vector-transfected SKOV3 or OV207 clones) and their HSulf-1-restored counterparts are treated with Iressa at a concentration previously shown to inhibit EGFR tyrosine phosphorylation (as determined in the experiments of Example 21) alone and in combination with several concentrations of cisplatin shown to induce apoptosis in 10-50% of HSulf-1-transfected cells. Likewise, the clones are treated with Iressa along with paclitaxel concentrations shown to induce apoptosis in 10-50% of HSulf-1-transfected cells (determined in the experiments of Example 17). Each signal transduction inhibitor is combined with each cytotoxic drug in the cell lines in a pairwise fashion, resulting in 16 combinations, each assessed in two HSulf-1-deficient clones and two HSulf-1-restored clones. For each experiment, apoptosis is scored as described in Example 17.

For each combination, the LD₅₀ is determined in the absence and presence of the signal transduction inhibitor in each cell line. The ratio of these two values in a particular cell line is the “dose modifying effect” of the signal transduction inhibitor. The data analysis approach described in Example 17 is used to determine whether the signal transduction inhibitors sensitize the cell lines and whether HSulf-1-transfected cells are preferentially sensitized.

### Example 23

### Effect of Signal Transduction Inhibitors Alone or in Combination in Xenografts

CD-1/NU mice bearing HSulf-1-deficient and HSulf-1 expressing xenografts on opposite flanks are randomly assigned to groups for treatment. If Iressa or CI-1040 is tested as a single agent, previously published doses of 150 mg/kg of each are administered (Sebolt-Leopold et al. (1999) Nature Med. 5:810-816; and Anderson et al. (2001) Int. J. Cancer 94:774-782). Likewise, data regarding the appropriate dose of BAY 37-9751 or an Akt inhibitor (preferably one that is close to clinical trials) are utilized to design single-agent experiments. Data regarding appropriate dosing of Iressa with cisplatin or paclitaxel in mice (Sirotnak et al. (2000) Clin. Cancer Res. 6:4885-4895) as well as CI-1040 with various agents are utilized to design combination trials involving these agents.

The endpoint is the time at which tumors reach three times their initial volumes. HSulf-1-deficient and HSulf-1-containing clones grafted onto the same mouse are compared. Once these variables are summarized by treatment group, the growth delays resulting from various single agents (e.g., Iressa or CI-1040) or from certain combinations (e.g., Iressa+cisplatin vs. cisplatin alone) are compared using survival methods and Wilcoxin rank tests.

### Example 24

### Materials and Methods for HCC Studies

Tumor Samples: Thirty-one HCC tumors were used for the real-time PCR experiments and 94 HCCs for the LOH experiments. Tumor samples with matched adjacent benign tissue were collected during surgical resections at the Mayo Clinic between 1991 and 2001, frozen in liquid nitrogen, and stored at −80° C. Sections from each specimen were examined by a pathologist and graded histologically.

HCC Cell Lines: The following 11 HCC cell lines were obtained from the ATCC and cultured as recommended by the ATCC: HepG2, Hep3B, Huh-7, PLC/PRF/5, SK-Hep-1, SNU182, SNU387, SNU398, SNU423, SNU449, and SNU475.

Isolation of total RNA, semi-quantitative RT-PCR, and quantitative real-time PCR: Total RNA was extracted from 31 pairs of matched HCCs and adjacent benign liver tissue and the 11 HCC cell lines using the RNEASY® kit (Qiagen). cDNA synthesis was performed using SUPERSCRIPT® II RNase H reverse transcriptase (Invitrogen/Life Technologies) to transcribe 1-5 μg of total RNA primed with 1 μl of 500 μg/ml random hexamers. Primers used for semi-quantitative RT-PCR were: hSulf1-F (5′-GAGCCATCTTCACCCATTCAAG-3′; SEQ ID NO:41) and hSulf1-R (5′-TTCCCAACCTTATGCCTTGGGT-3′; SEQ ID NO:14), yielding an 826 bp PCR product; GAPDH-F (5′-ACCACAGTCCATGCCATCAC-3′; SEQ ID NO:15) and GAPDH-R (5′-TCCACCACCCTGTTGCTTGTA-3′; SEQ ID NO:16). PCR reaction mixes contained 50 mM KCl, 10 mM Tris-HCl (pH 8.3), 1.5 mM MgCl₂, 400 nM each of forward and reverse primers, dNTPs (PE Biosystems, Foster City, Calif.), and 1 U Taq DNA polymerase (Invitrogen Corp., Carlsbad, Calif., USA). The PCR procedure included denaturation at 94° C. for 5 minutes followed by 34 cycles of 30 seconds at 94° C., 30 seconds at 62° C., and 30 seconds at 72° C., followed by an extension at 72° C. for 10 minutes. For quantitative real-time PCR analysis of HCC tumors, the hSulf1 primers were hSulf1RT-F (5′- CCACCTACCACTGTCCGAGT-3′; SEQ ID NO:39) and hSulf1RT-R (5′-TCTGCCGTCTCTTCTCCTTC-3′; SEQ ID NO:40), yielding a 379 bp PCR product. Real-time PCR was performed according to the manufacturer's recommendation in a Roche LightCycler using the following profile: 95° C. for 90 seconds followed by 34 cycles of 0 seconds at 95° C., 10 seconds at 61° C., and 20 seconds at 72° C. hSulf1 mRNA levels were normalized by comparison to 18S ribosomal RNA levels measured in the same samples. The 18S primers used were the Ambion Universal 18S PCR Primer Pair from the QuantumRNA 18S Internal Standards kit (Ambion Inc., Austin, Tex.). The profile for 18S real-time PCR was the same as for hSulf1 except that the annealing temperature was 60° C. Each measurement was performed in quadruplicate; a standard curve prepared from dilutions of synthesized hSulf1 and 18S standards was used to calculate the corresponding message levels. The ratio of the normalized hSulf1 mRNA expression in tumor/benign tissue was plotted on a log scale.

Loss of Heterozygosity (LOH) Analysis: DNA was extracted from 94 pairs of matched HCCs and adjacent benign liver tissue using the DNEASY® kit (Qiagen). Nine polymorphic markers spanning chromosome 8q were identified, including 6 markers from the hSulf1 gene region (Table 2). Each PCR reaction was performed in duplicate with fluorescently labeled oligonucleotide primers and 50 ng genomic DNA in a final PCR reaction volume of 20 μl. PCR amplification was performed for 35 cycles using 1.5 U Amplitaq Gold (PE Biosystems). PCR products were separated on an ABI 3100 DNA sequencer with the GeneScan 500 LIZ standard marker. Genotypes were analyzed using GeneScan 3.7 software. Samples were designated as informative (heterozygous) or non-informative (homozygous). For the informative samples a signal intensity ratio was determined between the tumor and its corresponding benign pair and according to the values obtained, the samples were scored as negative (no LOH) or positive (LOH)²¹.

Treatment with the DNA methylation inhibitor, 5-aza-2′-deoxycytidine. The hSulf1-negative HCC cell lines Huh7, SK-Hep-1, and SNU449 were maintained in medium containing 10% FBS and antibiotics as recommended by ATCC. Cells were seeded into six-well plates at 10⁵ cells per well. After overnight attachment, cells were cultured in the presence of 0, 5, or 10 μM 5-aza-2′-deoxycytidine for 5 days, harvested and subjected to RNA extraction using the RNEASY® mini kit (Qiagen). RNA (2 μg) was reverse transcribed as described above and semi-quantitative RT-PCR performed as described. Each experiment was performed 3 times.

Establishment of hSulf1 Stable Transfectant Clones: Plasmid vectors expressing either the N-terminal sulfatase domain (hSulf1-ΔC), the C-terminal portion (hSulf1-ΔN), or the full-length hSulf1 cDNA cloned into the GFP Fusion TOPO TA expression plasmid (Invitrogen) in the sense and antisense orientation were used (Lai et al. (2003) J. Biol. Chem. 278:23107-23117). Sulfatase negative SNU449, Hep3B, or Huh-7 cells were transfected using a mixture of hSulf1-expressing plasmid DNA or pcDNA3.1 vector DNA and LIPOFECTAMINE PLUS™ (Invitrogen/Life Technologies) reagent (Roberts et al. (1997) Gastroenterol. 113:1714-1726). The cells were placed under selective pressure in medium containing 400 μg/ml Geneticin (Invitrogen/Life Technologies) for 15-20 days. Geneticin resistant clones were isolated using cloning cylinders and transferred for expansion. Several stable clonal transfectants were generated from each cell line. Expression of hSulf1 by the stable clones was confirmed by semi-quantitative RT-PCR. Control clones transfected with pcDNA3.1 vector DNA were also selected. Transient transfections with antisense hSulf1-expressing plasmid vector were performed as above and cells were studied 48 hours after transfection.

Immunocytochemistry and confocal microscopy: For hSulf1 and FGFR1, SNU449 cells that were stably-transfected with a plasmid expressing FLAG®-epitope tagged hSulf1 were grown on glass coverslips for 24 hours, rinsed with Dulbecco's PBS (D-PBS) at room temperature and fixed for 20 minutes with 2.5% formaldehyde in PIPES buffer (0.1 M PIPES, 3 mM MgSO₄, 1 mM EGTA, pH 6.95). Cells were rinsed with D-PBS, blocked in 5% goat serum, 5% glycerol in D-PBS for 1 hour at 37° C., and incubated with anti-FLAG® monoclonal antibody (1:250; Sigma Chemical Co.) for 2 hours at 37° C. Cells were rinsed three times for 10 minutes each with D-PBS and incubated with FITC-labeled goat anti-mouse IgG (1:300; Molecular Probes, Eugene, Oreg.) for 1 hour at 37° C. Cells were then washed three times with D-PBS and mounted with DAPI on a glass slide (Cao et al. (1998) Mol. Bio. Cell 9:2595-2609). For co-localization of hSulf1-GFP with FGFR1, immunocytochemistry was performed as described using rabbit anti-FGFR1 antibody (Santa Cruz Biotechnology, Santa Cruz, Calif.) on SNU449 cells stably-transfected with a plasmid expressing an hSulf1-GFP fusion protein. Microscopy was performed with an Axiovert 35 Epifluorescence Microscope (Carl Zeiss Inc., Thornwood, N.Y.) and a Confocal Microscope (Zeiss LSM-510), with excitation at 488 nm and emission at 568 nm.

To examine sulfated cell surface HSGAGs, immunocytochemistry was performed on SNU449 cells stably-transfected with hSulf1 or with control plasmid vector as described above using a primary anti-mouse antibody that recognizes native heparan sulfate containing the N-sulfated glucosamine residue (10E4-mAb, 1:30 dilution, Seikagaku America). For control experiments, cells were incubated with heparitinase I (Sigma), followed by immunocytochemistry using an “anti-stub” antibody that recognizes HSGAG residues that are exposed by heparitinase action (3G10-mAb, Seikagaku).

Sulfatase assay: To assay sulfatase activity in whole cell extracts, equal numbers of cells (5×10⁶) were lysed in 1 ml of lysis buffer (10 mM Hepes, 150 mM NaCl, 1% NP-40, 10% glycerol, 1 mM PMSF and 1 mM EGTA) and the lysate incubated on ice for 10 minutes. 4-MUS was used as the substrate. Cell lysates with 100 μg protein were diluted with SIE (250 mM sucrose, 3 mM imidazole, 0.1% absolute ethanol, pH 7.4) to a total volume of 100 μl and aliquoted into duplicate 12×75 mm glass test tubes on ice. One hundred microliters of 1 μM 4-methylumbelliferylsulfate was added to each tube, mixed and incubated at 37° C. for 20 minutes. Two ml stop solution (50 mM glycine, 5 mM EDTA, pH 10.4) was then added, mixed and released 4-methylumbelliferone measured using a fluorometer (Sequoia-Turner model 450, Mountain View, Calif.; excitation wavelength 360 nm, emission wavelength 460 nm). Sulfatase activity was expressed as nanomoles of 4-methylumbelliferone per mg protein released per hour.

Immunoblot analyses of FGF2 and HGF signaling: For analysis of cell surface FGF receptor phosphorylation, plasma membranes were prepared as described by Hadac et al. ((1996) Pancreas 13:130-139). Nitrocellulose membrane blots were probed with mouse monoclonal anti-phospho-FGFR1 antibody, rabbit anti-FGFR1 antibody and mouse anti-β-actin antibody (all from Santa Cruz). Immunoblot analysis of whole-cell lysates for phospho-c-Met, phospho-ERK and total ERK was performed on cells lysed in Laemmli buffer without bromophenol blue. Blots were probed with rabbit anti-phospho-c-Met antibody, rabbit anti-phospho-p44/42 ERK antibody, and rabbit anti-total p44/42 ERK antibody (Cell Signaling). Immunoblots were developed using ECL enhanced chemiluminescence reagents after incubation with horseradish peroxidase-conjugated secondary antibodies (Amersham).

Cell proliferation assays: For cell counting assays, cells were plated on a 6-well plate at 10⁵ cells per well and incubated in 10% FCS or 0.25% FCS with or without 10 ng/ml FGF2 for up to 4 days. Viable cells identified by trypan blue exclusion were counted after each 24 hour period. For MTT assays, cells were plated on a 96-well plate at 3000 cells per well and incubated in 0.25% FCS with or without 10 ng/ml FGF2 for 48 hours. Cell viability was then assessed by MTT-reducing capacity (Betz et al. (2002) Phytochem. Photobiol. Sci. 1:315-319). The viability of untreated vector-transfected control cells was set to 100%, and the viability of FGF2-treated vector-transfected and untreated and treated hSulf1-transfected cells was expressed as a percentage of formazan absorbance compared with that of control cells. Each experiment was performed in six replicates at least three independent times.

Detection and quantitation of apoptosis: Apoptosis was quantitated by assessing nuclear changes indicative of apoptosis (i.e., chromatin condensation and nuclear fragmentation) using the DNA binding dye DAPI as described by Roberts et al. (supra). Cells were seeded in 35-mm plates at 2×10⁵ cells per well. After incubation for 24 hours, the plates were washed, changed to serum free medium containing 1 μM staurosporine, and incubated for 5 hours at 37° C. Five micrograms of DAPI were added and the plates were incubated for 20 minutes at room temperature in the dark. The cells were then examined by fluorescence microscopy (Nikon Eclipse TE200; Nikon Corp., Tokyo, Japan) using excitation and emission filters of 380 and 430 nm, respectively. For each treatment, at least 300 cells in six different high-power fields were counted. To determine whether apoptosis was occurring through a caspase mediated process, cells were pretreated with 40 μM of the caspase inhibitor Z-VAD(O-Me)-fmk (Sigma) for 1 hour before addition of staurosporine. For experiments using cisplatin, cells cultured in medium with 10% serum were treated with or without 5 μM cisplatin for 24 hours, then stained with DAPI and counted.

Flow cytometry also was used to evaluate apoptosis. After a 5 hour treatment with 1 μM staurosporine as described above, cells were collected by centrifugation and washed twice in ice-cold PBS with 3% heat-inactivated FBS and 0.02% sodium azide. Cells were stained with 50 μg/ml 7-AAD for 15 minutes in the dark. The cells were resuspended in 500 μl PBS and analyzed using a FACScan analyzer (BD Bioscience). Apoptotic cells were calculated as percent of 7-AAD positive cells in 5,000 or more cells per sample.

Immunoblot analysis for mitochondrial cytochrome c release: Immunoblot analysis for cytochrome c utilized cytosolic extracts prepared by selective digitonin permeabilization (Leist et al. (1998) Mol. Pharmacol. 54:789-801). Blots were probed with mouse monoclonal anti-cytochrome c antibody (BD Pharmingen) or mouse anti-β-actin antibody. For caspase 9, whole-cell lysates were prepared as described above for the sulfatase assay. Membranes were probed with mouse anti-procaspase 9 or mouse anti-β-actin antibody. Immunoblots were developed using ECL enhanced chemiluminescence reagents after incubation with horseradish peroxidase-conjugated secondary antibodies.

Statistical Analysis: All data represent at least three independent experiments using cells from separate cultures and are expressed as the mean±SEM. Differences between groups were compared using an unpaired two-tailed t test.

### Example 25

### Expression of hSulf1 mRNA in Primary HCCs and HCC Cell Lines

As described herein, hSulf1 is down regulated in 77% (23/30) of ovarian carcinomas, and in the majority of cancer cell lines of ovarian, breast, pancreas, kidney and liver origin. To determine whether hSulf1 also was down regulated in primary HCCs, hSulf1 expression was evaluated in 31 primary HCCs by quantitative real-time PCR. Twenty-two of the HCCs were randomly selected and 9 additional tumors were selected based on known LOH at the hSulf1 locus. Of the 31 total HCCs examined, 9 (29%) showed decreased hSulf1 mRNA expression by real time PCR. These included 5 of the 22 randomly selected HCCs (23%), and 4 of the 9 HCCs with known LOH (44%). As the availability of cell lines would facilitate exploration of the significance of hSulf1 loss in HCC, the evaluation of hSulf1 expression was extended to a total of 11 HCC cell lines (HepG2, Hep3B, Huh-7, PLC/PRF/5, SK-Hep-1, SNU 182, SNU387, SNU398, SNU423, SNU449, and SNU475) using semi-quantitative RT-PCR. Nine of the 11 HCC cell lines (82%) displayed low or undetectable levels of hSulf1 mRNA, while the other 2 cell lines (SNU182 and SNU475) expressed high levels of the hSulf1 mRNA.

### Example 26

### LOH Analysis at the hSulf1 Locus in HCCs

To determine the mechanism of down regulation of hSulf1 in HCCs, allelic imbalance at the hSulf1 locus was assessed by LOH analysis. Nine polymorphic microsatellite markers flanking the hSulf1 gene were identified (Table 2). Analysis of 94 primary HCC tumors revealed that LOH of markers surrounding hSulf1 ranged from 25-42%, with the peak of 42% immediately centromeric to the hSulf1 gene. Of the 31 HCC tumors in which hSulf1 expression was assessed by real-time PCR, 14 showed LOH at the hSulf1 locus. Seven of the 14 tumors with LOH (50%) also showed down regulation of hSulf1 mRNA expression.

TABLE 2
LOH surrounding the hSulf1 locus on chromosome 8q in HCC studies

| | | | % LOH |
|---|---|---|---|
| | | | (# samples |
| | | | with LOH/# |
| Microsatellite | Distance from | Chromosomal | informative |
| marker | hSulf1 gene | band | samples) |
| D8S543 | 370 Kb centromeric | 8q.13.3 | 32 (20/63) |
| HS1-1 | 55 Kb centromeric | 8q13.3 | 27 (22/82) |
| D8S381 | 39 Kb centromeric | 8q13.3 | 33 (25/76) |
| HS1-2 | 25 Kb centromeric | 8q13.3 | 42 (22/52) |
| HS1-3 | 42 Kb telomeric | 8q13.3 | 25 (9/36) |
| D8S1795 | 193 Kb telomeric | 8q13.3 | 25 (15/59) |
| D8S1760 | 10 Mb telomeric | 8q21.13 | 27 (21/79) |
| D8S1842 | 60 Mb telomeric | 8q24.21 | 27 (19/71) |
| (c-Myc) | | | |
| D8S1925 | 76 Mb telomeric | 8q24.3 | 24 (17/72) |
| (telomere) | | | |

### Example 27

### HCC Cell Lines Respond to Inhibition of DNA Methylation

Down-regulation of tumor suppression proteins frequently occurs through hypermethylation of regulatory sequences. To investigate the potential role of methylation in regulation of hSulf1 expression, the hSulf1-negative cell lines, SNU449 and Huh-7 were treated with increasing concentrations of 5-aza-2′-deoxycytidine, a DNA methylase inhibitor. The SNU449 and Huh-7 cell lines exhibited reactivation of hSulf1 expression in response to the 5-aza-2′-deoxycytidine treatment.

### Example 28

### hSulf1 is Localized at the Cell Surface of HCC Cell Lines

The hSulf1 sequence contains a hydrophilic domain homologous to that of the recently identified quail sulfatase, Qsulf1 (Dhoot et al. (2001) Science 293:1663-1666). Dhoot et al. showed that a mutant of Qsulf1 with a deletion of the hydrophilic domain was released into the culture medium, suggesting that Qsulf1 is docked to the cell surface through interactions of the hydrophilic domain with cell surface components. To determine the subcellular localization of hSulf1 in HCC cells, immunocytochemistry was performed on non-permeabilized cells transiently transfected with a plasmid expressing FLAG®-tagged hSulf1 under control of the CMV promoter. An anti-FLAG® antibody was used for staining, followed by fluorescence microscopy. The FLAG®-tagged hSulf1 was localized to the cell surface. Cells transfected with an hSulf1-GFP plasmid also were examined with immunocytochemistry using an anti-FGFR1 antibody, which recognizes cell surface FGF receptors. These experiments also showed co-localization of hSulf1 with FGFR1 at the cell surface. Thus, hSulf1 was localized to the cell surface of HCC cells, in the same subcellular compartment as HSGAGs.

### Example 29

### hSulf1 Expression is Associated with a Decreased Amount of Sulfated HSGAGs

To determine whether hSulf1 expression causes desulfation of cell surface HSGAGs, immunocytochemistry was performed using the 10E4 anti-HSPG monoclonal antibody, which recognizes native heparan sulfate containing the N-sulfated glucosamine moiety (David et al. (1992) J. Biol. Chem. 119:961-975; Bai et al. (1994) J. Histochem. Cytochem. 42:1043-1054; Nackaerts et al. (1997) Int. J. Cancer 74:335-345). SNU182, which expresses a high level of endogenous hSulf1, was compared to parental SNU449 cells or SNU449-Vector cells, which do not express hSulf1, and to SNU449-hSulf1-1, SNU449-hSulf1-2 and SNU449-hSulf1-3, three stable clones expressing full-length hSulf1. The parental SNU449 and SNU449-Vector cells showed cell surface staining for N-sulfated glucosamine-containing HSGAGs, while the cell surface staining was diminished or absent in the SNU182 cell line and all three SNU449-hSulf1 clones. The 3G10 “anti-stub” antibody was used after heparitinase I treatment to confirm the presence of HSPG stubs on both sulfatase expressing and sulfatase-negative cell lines. Transient expression of a construct expressing antisense hSulf1 mRNA restored the cell surface10E4 anti-HSPG immunoreactivity of the sulfatase-positive cell lines. These results strongly suggested that hSulf1 desulfates HSGAGs at the cell surface.

### Example 30

### hSulf1 Sulfatase Activity is Mediated by the N-Terminal Sulfatase Domain

To determine whether the putative sulfatase domain of hSulf1 is enzymatically active in HCC, sulfatase activity was assayed in extracts prepared from the SNU182 and SNU475 HCC cell lines, which express high levels of hSulf1 mRNA, and from the SNU449 cell line, which does not express hSulf1 at detectable levels. Cell lines developed by stable transfection of SNU449 with either a vector control (SNU449-Vector) or plasmids containing the N-terminal sulfatase-domain containing region (SNU449-hSulf1-ΔC), the C-terminal region (SNU449-hSulf1-ΔN), or the full-length hSulf1 cDNA (SNU449-hSulf1-1) also were assayed. 4-MUS (Sigma) was used as the substrate for sulfatase activity. Extracts were pretreated with the steroid sulfatase inhibitor estrone-3-O-sulfamate (EMATE; Sigma; Purohit et al. (1995) Biochem. 34:11508-11514) prior to assaying sulfatase activity.

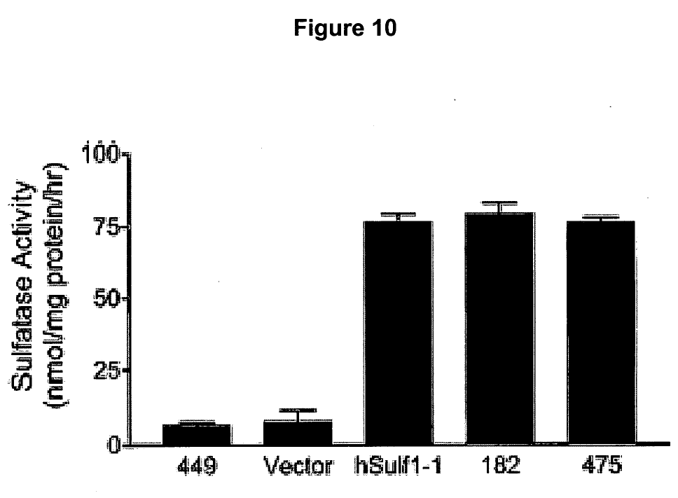

These studies showed that non-steroid sulfatase activity was low in parental SNU449 and SNU449-Vector cells, but considerably higher in SNU182, SNU475, and stably-transfected SNU449-hSulf1-1 cells (p<0.05, FIG. 10). Further, extracts from the sulfatase-domain expressing SNU449-hSulf1-ΔC cell line had almost the same level of sulfatase activity as the cell line expressing full-length hSulf1, while extracts from the C-terminal expressing SNU449-hSulf1-ΔN cell line had about the same level of sulfatase activity as the low-expressing parental SNU449 and SNU449-Vector cell lines. The stably-transfected hSulf1 clones SNU449-hSulf1-2 and SNU449-hSulf1-3 also showed increased levels of sulfatase activity, similar to that of SNU449-hSulf1-1. In the SNU449-hSulf1-1 cell line, most of the sulfatase activity after EMATE treatment was presumably due to hSulf1, while other sulfatases may contribute to the sulfatase activity noted in SNU182 and SNU475.

### Example 31

### hSulf1 Decreases Signaling by FGF2 and HGF

To explore the role of desulfation of cell surface HSGAGs by hSulf1 in cellular growth control, the effect of hSulf1 expression on FGF2 and HGF signaling was investigated in HCC cell lines with or without hSulf1 expression. As described above, formation of the FGF2-HSGAG-FGFR ternary complex induces receptor dimerization and activation of the intracellular FGFR tyrosine kinase, which phosphorylates the receptor and also activates cellular signaling pathways including the MAPK pathway. MAPK activation leads to phosphorylation of p44/42 (ERK1/2), which is required for cell proliferation. Thus, phosphorylation of FGFR and p44/42 was evaluated to assess FGF2 and HGF signaling in various HCC lines.

SNU449-Vector cells showed increased FGFR1 phosphorylation after FGF2 treatment, whereas SNU449-hSulf1-1 cells showed essentially no change in FGFR1 phosphorylation. Parental SNU449 and SNU449-Vector cells showed sustained phosphorylation of p44/42 ERK. In contrast, three different hSulf1-expressing SNU449-hSulf1 clones showed low baseline p44/42 ERK phosphorylation and little or no sustained p44/42 ERK phosphorylation in response to FGF2 treatment. hSulf1-expressing Huh-7-hSulf1-1 and Hep3B-hSulf1-1 cells also showed less p44/42 ERK phosphorylation at baseline and in response to FGF2 treatment than Huh-7-Vector and Hep3B-Vector cells. Parental SNU182 cells, which express high levels of endogenous hSulf1, showed essentially no activation of p44/42 ERK in response to FGF2. Similarly, SNU449-hSulf1 cells showed significantly less c-Met and p44/42 ERK phosphorylation in response to HGF treatment than SNU449-Vector cells. Thus, hSulf1 expression decreases signaling by both FGF2 and HGF through the MAPK pathway.

### Example 32

### Expression of hSulf1 Inhibits FGF2-Mediated Proliferation of HCC Cells

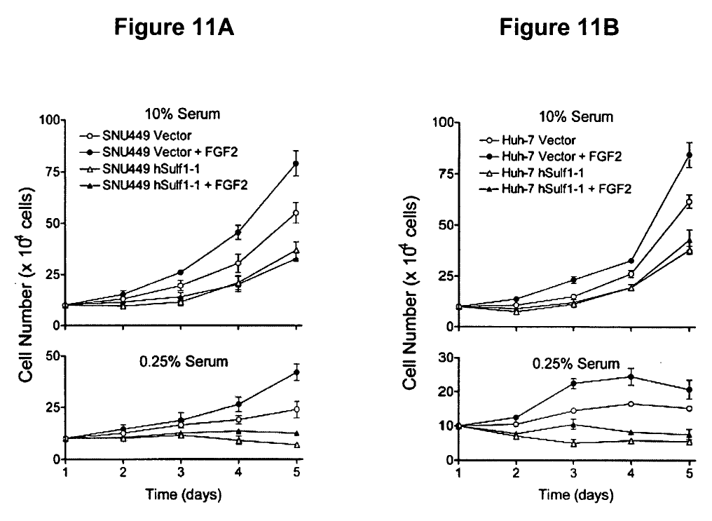

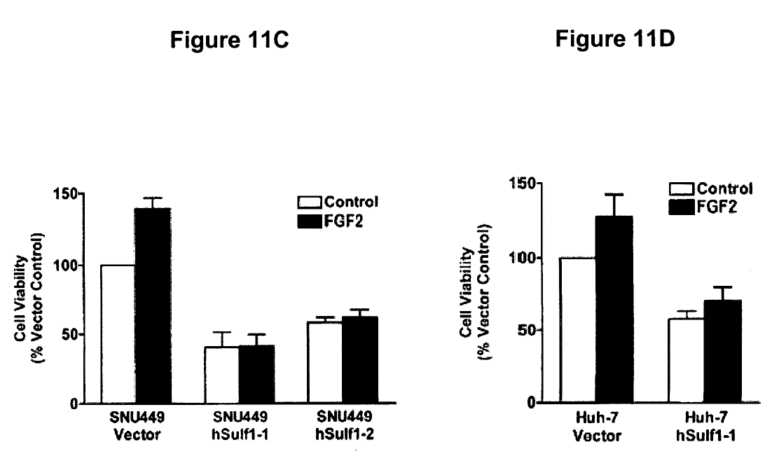

FGF2 is a potent mitogen for primary hepatocytes and is frequently expressed at high levels in HCCs. To determine whether inactivation of hSulf1 expression in HCC cells leads to an increased sulfation state of cell surface HSGAGs and potentiates FGF2 signaling, resulting in increased cellular proliferation, the effect of stable hSulf1 transfection into the SNU449 (hSulf-negative) cell line was assessed. FGF-induced cell proliferation was measured using trypan blue exclusion and the cell viability MTT assay. Vector-transfected SNU449-Vector cells and the stably-transfected hSulf1 clones SNU449-hSulf1-1, SNU449-hSulf1-2, and SNU449-hSulf1-3 were plated in either 10% serum or 0.25% serum. Cells were incubated in the presence or absence of FGF2, and cell growth was measured by counting viable cells or by the MTT assay. Vector-transfected SNU449 cells showed increased growth in response to FGF2 (FIGS. 11A and 11C). Expression of hSulf1 led to almost complete abrogation of FGF-dependent cell growth in all three SNU449-hSulf1 clones examined. In the limiting 0.25% serum concentration, the number of hSulf1-transfected cells on plates not treated with FGF2 was decreased, suggesting that at limiting concentrations of growth factors, hSulf1 expression reduces cell viability. To confirm that the effect of hSulf1 was not limited to the SNU449 cell line, similar experiments were performed using vector and hSulf1-transfected Huh-7 clones, with essentially identical results (FIGS. 11B and 11D).

### Example 33

### Cell Lines with High hSulf1 Activity are More Sensitive to Induced Apoptosis

The effect of hSulf1 expression on the sensitivity of HCC cells to apoptosis induced by staurosporine was examined. Confirmation that cell death occurred by apoptosis was obtained using (a) fluorescence microscopy after staining with DAPI, (b) flow cytometry after staining with 7-AAD to detect cells in the early stages of apoptosis, and (c) agarose gel electrophoresis of DNA from cells treated with staurosporine, which showed the characteristic ladder pattern of internucleosomal DNA cleavage. In addition, experiments were conducted to show that staurosporine-induced apoptosis of hSulf1-expressing cells was characterized by release of mitochondrial cytochrome c into the cytosol and by activation of procaspase 9.

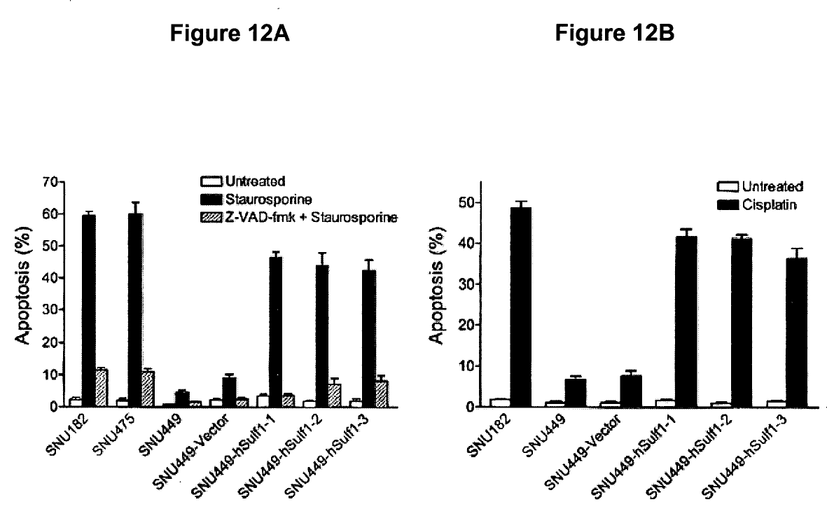

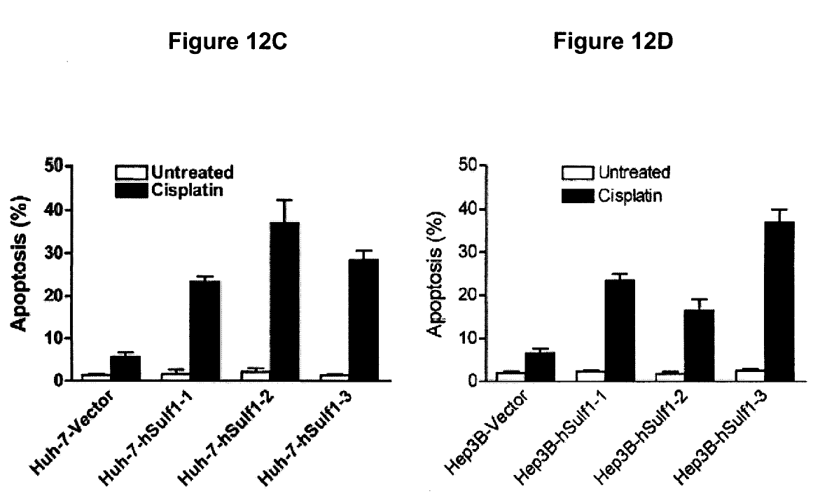

Staurosporine induced a large increase in apoptosis in the high hSulf1-expressing cell lines SNU182 and SNU475, but not in the hSulf1-negative cell line SNU449 (FIG. 12A). To show that this was not due simply to differences in phenotype of the cell lines, apoptosis was assessed in the stably-transfected hSulf1 clones SNU449-hSulf1-1, SNU449-hSulf -2, and SNU449-hSulf1-3, which express the full-length hSulf1 protein. All three hSulf1-transfected clones showed a high sensitivity to staurosporine-induced apoptosis, similar to the endogenously high hSulf1-expressing cell lines SNU182 and SNU475. In contrast, the hSulf1 negative parental cell line SNU449 and vector-transfected SNU449-Vector cells showed minimal sensitivity to staurosporine-induced apoptosis (FIG. 12A). Thus, expression of hSulf1 increased the sensitivity of HCC cells to apoptosis. To confirm that hSulf1- mediated apoptosis occurred through a classical caspase-mediated process, cells were pretreated with 40 μM of the caspase inhibitor Z-VAD(O-Me)-fmk for 1 hour before addition of staurosporine. Z-VAD(O-Me)-fmk inhibited staurosporine-induced apoptosis, confirming the requirement for caspase activation in hSulf1-mediated apoptosis.

To evaluate the potential relevance of hSulf1 expression to chemotherapy-induced apoptosis, cell lines were treated in the presence or absence of 5 μM cisplatin. The cell line endogenously expressing hSulf1 (SNU182) and all three hSulf1-transfected stable clones showed a high sensitivity to cisplatin-induced apoptosis. In contrast, the hSulf1 negative parental SNU449 and SNU449-Vector cells were resistant to cisplatin-induced apoptosis (FIG. 12B). Similar experiments with Hep3B and Huh-7-derived Vector and hSulf1-transfected stable clones confirmed the effect of hSulf1 expression in increasing sensitivity of cell lines to cisplatin-induced apoptosis (FIGS. 12C and 12D).

### Example 34

### hSulf1 Promotion of Staurosporine-Induced Apoptosis is Dependent on Expression of an Intact N-terminal hSulf1 Sulfatase Domain

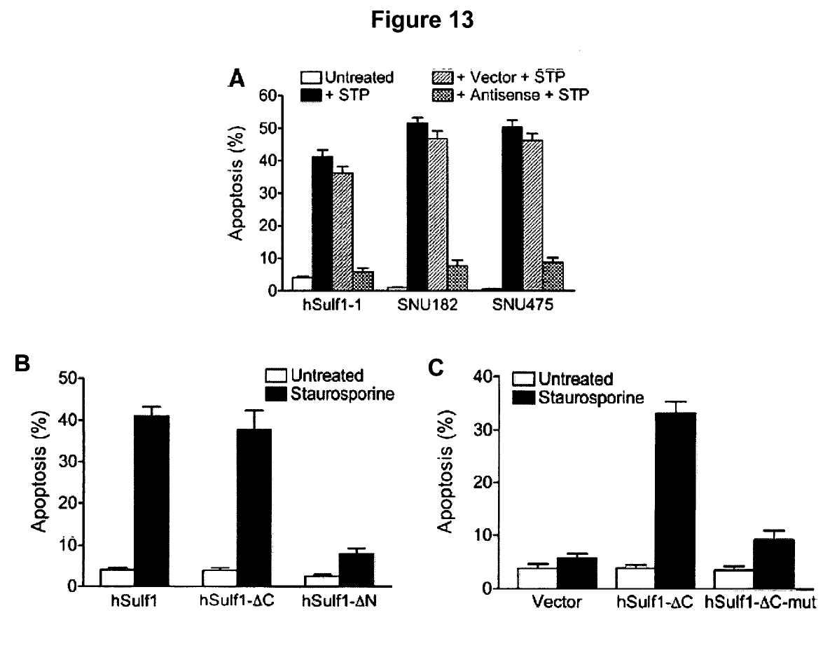

To determine whether the sensitivity of hSulf1-expressing cell lines to staurosporine-induced apoptosis was dependent on the sulfatase activity of the hSulf1 protein, the SNU449-hSulf1-1 cell line and the SNU182 and SNU475 cell lines (which express high levels of hSulf1) were transiently transfected with either an empty vector or a plasmid expressing an antisense hSulf1 sequence. Transfection efficiencies as determined by co-transfection with GFP-expressing constructs were 80-90%. Cells were treated with 1 μM staurosporine to induce apoptosis. Vector-transfected cells showed no difference in apoptosis from the untransfected cell lines, while apoptosis was significantly inhibited in antisense hSulf1 transfected cells (FIG. 13A). Antisense hSulf1-transfected cells also showed increased p44/42 ERK phosphorylation in response to FGF2, confirming the downstream effect of abrogation of hSulf1 expression. Next, apoptosis was assessed in SNU449 cells transiently-transfected with plasmids expressing either the sulfatase domain-containing N-terminal region of hSulf1 (SNU449-hSulf1-ΔC) or the C-terminal region of hSulf1 (SNU449-hSulf1-ΔN). SNU449-hSulf1-ΔC cells showed a high sensitivity to staurosporine-induced apoptosis, similar to the high hSulf1-expressing cell lines SNU182 and SNU475. In contrast, SNU449-hSulf1-ΔN cells showed minimal sensitivity to staurosporine-induced apoptosis, similar to the hSulf1 negative parental cell line SNU449 (FIG. 13B). Finally, the codons for the two conserved cysteines in the catalytic site of the sulfatase domain were mutated in the SNU449-hSulf1-ΔC plasmid. The resulting plasmid, designated SNU449-hSulf1-ΔC-mut, was transiently transfected into SNU449 cells. Transfection of SNU449-hSulf1-ΔC resulted in an increase in measurable sulfatase activity in SNU449 cells, whereas transfection of SNU449-hSulf1-ΔC-mut resulted in no change in measurable hSulf1 sulfatase activity. Mutation of the sulfatase domain also abrogated the increase in sensitivity to apoptosis (FIG. 13C). Thus, the ability of hSulf1 to increase the sensitivity of HCC cells to staurosporine-induced apoptosis is dependent on the presence of an intact, presumably active, N-terminal sulfatase domain within the hSulf1 protein.

### Example 35

### Materials and Methods for SCCHN Studies

Cell culture: Three head and neck cancer cell lines, 012SCC, WMMSCC (Strome et al. (2002). Clin. Cancer Res. 8:281-286) and 015SCC, were obtained from ATCC and cultured as recommended.

Drugs and Reagents: Staurosporine (Sigma) was dissolved in DMSO at a concentration of 1 mM, stored at −20° C., and subsequently diluted with serum-free medium before use. In all experiments the concentration of DMSO did not exceed 0.1%. Cisplatin (Sigma) was prepared immediately before use as a 1000-fold concentrated solution in DMSO.

Semi-quantitative RT-PCR: Total RNA was extracted from 3 SCCHN cell lines using the RNEASY® mini kit (Qiagen). cDNA synthesis was performed as described (Shridhar et al. (2002) supra). Reverse transcribed cDNA (50-100 ng) was used in a multiplex reaction with Sulf-3F (5′-GAGCCATCTTCACCCATTCAA-3′; SEQ ID NO:13), Sulf-3R (5′-TTCCCAACCTTATGCCTTGGGT-3′; SEQ ID NO:14) and GAPDH-F (5′-ACCACAGTCCATGCCATCAC-3′; SEQ ID NO:15) and GAPDH-R (5′-TCCACCACCCTGTTGCTTGTA-3′; SEQ ID NO:16) in separate reactions to yield 760 bp, 1260 bp and 825 bp products, respectively. The PCR reaction mixes contained 50 mM KCl, 10 mM Tris-HCl (pH 8.3), 1.5 mM MgCl₂, 400 μM each primer for HSulf-1 and 50 μM each primer for GAPDH, and 0.5 units of Taq polymerase (Promega) in a 12.5 μl reaction volume. The conditions for amplification were: 94° C. for 3 minutes followed by 29 cycles of 94° C. for 30 seconds, 58° C. for 30 seconds, and 72° C. for 30 seconds in a Perkin Elmer-Cetus 9600 Gene-Amp PCR system. The products of the reaction were resolved on a 1.6% agarose gel and photographed using the Gel Doc 1000 photo documentation system.

Establishment of stable transfectants: Exponentially growing 012SCC cells in 100 mm dishes were washed with serum-free medium and treated with a mixture containing 4 μg plasmid, 30 μl LIPOFECTAMINE™, and 20 μl PLUS™ reagent (Invitrogen/Life Technologies). After a 3 hour incubation, medium with serum was added. G418 (400 μg/ml) was added 24 hours later to select transfectants. Individual colonies were subsequently cloned using cloning cylinders. For controls, cells were similarly transfected with vector (pcDNA3.1 GFP-CT), and stable clones selected.

Sulfatase assay: Confluent flasks of stable transfectants were washed in ice cold PBS and lysed in SIE buffer (250 mM sucrose, 3 mM imidazole, pH 7.4, 1% ethanol) containing 1% (w/v) Nonidet P-40 and protease inhibitor cocktail (Roche Molecular Biochemicals). After cells were sheared by passage through a 27 gauge needle, protein concentrations were determined using the Bradford assay. 100 μg of total cellular protein was preincubated with 10 μM estrone-3-O-sulfamate (Sigma Chemicals) at 37° C. for 1 hour to inhibit steroid sulfatases. 4-MUS was then added to a final concentration of 10 mM in the presence of 10 mM lead acetate, in a total volume of 200 μl. After incubation for 24 hours at 37° C., the reaction was terminated by addition of 1 ml 0.5 M Na₂CO₃/NaHCO₃, pH 10.7. The fluorescence of the liberated 4-methylumbelliferone was measured using excitation and emission wavelengths of 360 nm and 460 nm, respectively.

Treatment with FGF-2 and HGF: To assess the role of HSulf-1 in FGF-2/HGF mediated signaling, vector-transfected and HSulf-1 clones 1 and 2 were serum starved for 8-12 hours and treated with diluent, 2 ng/ml FGF-2 (Sigma), or 5 ng/ml HGF (Research Diagnosis Inc, Flanders, N.J.) for 15 or 60 minutes. Following treatment, cells were rinsed with ice cold PBS, scraped from the dishes, and lysed at 4° C. in SDS sample buffer without bromophenol blue. Protein concentrations were determined with bicinchoninic acid (Pierce, Rockford, Ill.).

Immunoblotting: Equal amounts of protein (20 μg/lane) were separated by electrophoresis on a SDS gel containing a 4-12% SDS polyacrylamide gradient, and electrophoretically transferred to nitrocellulose. Blots were washed once with TBS-0.2% Tween 20 (TBST) and blocked with TBST containing 5% non-fat dry milk for 1 hour at 20° C. The blocking solution was replaced with a fresh solution containing 1:500 dilution of rabbit anti-phospho-ERK or anti-phospho-AKT-ser 473 (Cell Signaling Inc.). After overnight incubation at 4° C., the blots were washed three times for 10 minutes each in TBS/0.1% (w/v) Tween 20, and incubated with horseradish peroxidase-conjugated secondary antibody in 5% milk/TBST at 20° C. for 1 hour. After washing 3 times in TBST, the proteins were visualized using enhanced chemiluminescence (Amersham). The blots were stripped and reprobed with antisera that recognize total ERK or total Akt (Cell Signaling Inc.), antityrosine antibody py20, and anti c-Met antibody (Santa Cruz Biotech).

Cell Proliferation Assay: Cell growth was assessed using the MTT [3-(4,5-dimethylthiazol-2-yl)-2,5-diphenyl-2H-tetrazolium bromide] assay (Betz et al. supra). Three thousand cells of HSulf-1 012SCC clones 1 and 2 as well vector transfected 012SCC were plated in 96-well plates, and incubated at 37° C., 5% CO₂. At 24, 48, 72, and 96 hours, the medium was replaced with serum-free RPMI-1640 containing 0.2 mg/ml MTT and incubated for an additional 4 hours. Following this procedure, the wells were drained and 160 μl of DMSO was added per well to solubilize the MTT-formazan. After lightly vortexing the plate on an orbital shaker, the absorbance was read on a microplate reader (Bio Rad Model 3550-UV) using a measurement wavelength of 570 nm. Each individual experiment was performed at least three times and in six wells each.

[³H] Thymidine Incorporation Assay: Stable clonal cells were seeded into 24-well plates at a density of 10,000 cells per well and incubated overnight in the complete medium. The next day, the medium was replaced with the serum-free medium for 6 hours. Cells were then incubated with 0.5 Ci of ³H-thymidine for 24 hours. At the end of the incubation, the cells were washed 3 times with PBS-1 M thymidine and incorporated radioactivity was determined in TCA-precipitable fraction (Chien and Shah (2001) Int. J. Cancer 91:46-54).

Invasion Assay. Motility and invasion assays were performed in 6.5 mm diameter Transwell chambers (Costar, Cambridge, Mass.) with porous polycarbonate membranes (8.0 μm pore size). In both experiments, after 6 hours of serum starvation, the cells (10⁴/each well) were seeded on the upper side of the filter and recombinant HGF (40 ng/ml) was added to the lower chambers in serum free media. For invasion assay, BD Bio Coat Matrigel Invasion chambers were used (BD Bioscience; Clontech, Bedford, Mass.). After 4 hours for the motility assay or 24 hours for the invasion assay, cells on the upper side of the filters were mechanically removed. Cells that migrated to the lower side were either fixed with 4% paraformaldehyde and stained with 0.25% Coomassie blue for motility assays, or fixed with 100% methanol and stained with 1.0% methyl violet for invasion assay. The filters were photographed and cells were counted.

Analysis of Apoptosis: Apoptosis was quantitated by assessing the number of cells containing nuclear changes indicative of apoptosis (chromatin condensation and nuclear fragmentation) after staining with DAPI. HSulf-1 transfected 012SCC cells were seeded in 35-mm plates at a density of 2×10⁵ cells/well. After incubation at 37° C. for 24 hours, the plates were washed and changed to serum-free medium. Staurosporine or cisplatin was added to final concentrations of 1 μM and 5 μM, respectively. After a 5 hour incubation for staurosporine and a 24 hour incubation for cisplatin at 37° C., DAPI was added to each well at a final concentration of 5 μg/ml. After a 20 minutes incubation in the dark at 37° C., cells were examined by fluorescence microscopy (Nikon Eclipse TE200; Nikon Corp., Tokyo, Japan) using excitation and emission filters of 380 and 430 nm. An individual blinded to the experimental conditions counted at least 300 cells in six different high-power fields for each treatment. Each treatment was repeated at least three times, performed in triplicate each time. The significance of differences between experimental variables was determined using the Student t test.

Detection of Apoptosis by Flow Cytometry: HSulf-1-1 or vector transfected 012SCC cells were collected by centrifugation from untreated control and staurosporine treated cells. The cell pellets were washed twice in 4° C. buffer solution (PBS with 3% of heat-inactivated fetal bovine serum and 0.02% of sodium azide), and then were stained with 7-AAD (50 μg/ml) for 15 minutes in the dark. The cells were then resuspended in 500 μl of PBS and analyzed by FACScan (BD Bioscience). The percentage of apoptotic cells was presented as % of 7-AAD positive cells in a total of 10,000 cells for each sample.

Statistical Analysis: All data represent at least three independent experiments using cells from separate cultures and are expressed as the mean±SEM. Differences between groups were compared using an unpaired two-tailed t test.

### Example 36

### HSulf-1 Expression in SCCHN Cell Lines

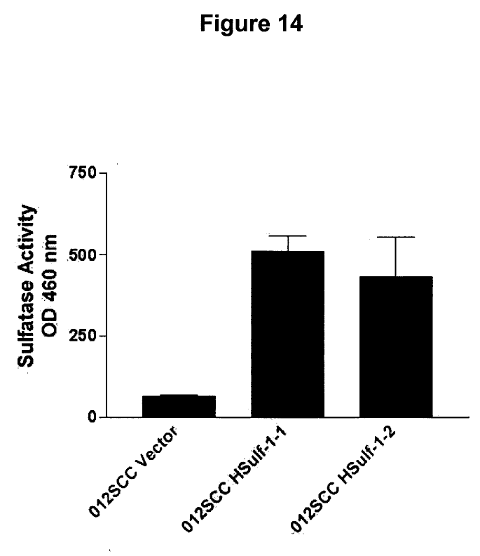

Semi-quantitative RT-PCR with primers Sulf 3F and 3R revealed a complete absence of HSulf-1 expression in all three SCCHN cell lines compared to a normal squamous epithelial control from the head and neck region from a patient with no signs of cancer. To assess the functional consequences of loss of HSulf-1 expression in SCCHN cell lines, full length (FL) HSulf-1 cDNA was transfected into 012SCC cell line and two stable clones expressing HSulf-1 (HSulf-1-1 and HSulf-1-2) were isolated. To confirm that the stable clones displayed sulfatase activity, sulfatase activity was measured using the fluorogenic substrate 4-MUS in the presence of the estrone sulfatase inhibitor EMATE as described in Example 35. There was an increase in the sulfatase activity in 012SCC HSulf-1-1 and HSulf-1-2 clones compared to the vector transfected control (FIG. 14). This activity was comparable in stably transfected HSulf-1 clones of ovarian cancer cell lines as disclosed herein.

### Example 37

### HSulf-1 Modulates FGF-2 Signaling

Experiments using sulfation specific antibodies revealed that HSulf-1 regulated HSPG sulfation in SCCHN cells. To explore the role that desulfation of cell surface HSGAGs by HSulf-1 lays in cellular growth control, the effect of HSulf-1 expression on FGF-2 signaling was investigated in SCCHN cell lines. Treatment with 2 ng/ml FGF-2 for 15 and 60 minutes resulted in a sustained ERK phosphorylation in vector-transfected clone, but a significant down regulation of phosphorylation in HSulf-1 expressing 012SCC clones 1 and 2. Transient transfection of WMMSCC cells with FL-HSulf-1 dampened FGF signaling as assessed by activation of ERK p42/44, whereas mutation of two conserved cysteines at the active site of the sulfatase domain only construct abrogated this modulation. This is similar to what was observed in ovarian tumor cell lines (above).

### Example 38

### HSulf-1 Modulates HGF Mediated ERK and PI3K/Akt Signaling

Since the heparin binding HGF mediated signaling seems to play an important role in head and neck cancer, the effect of HSulf-1 expression in SCCHN clones was evaluated. Treatment with 5 ng/ml HGF for 15 and 60 minutes resulted in a sustained c-Met phosphorylation in vector transfected 012SCC clone, but a significant down regulation of phosphorylation in HSulf-1 expressing 012SCC clones 1 and 2. This down regulation was reflected in activation of both Akt and ERK phosphorylation in HSulf-1 expressing clones. Total c-Met, total Akt and total MAPK served as loading controls.

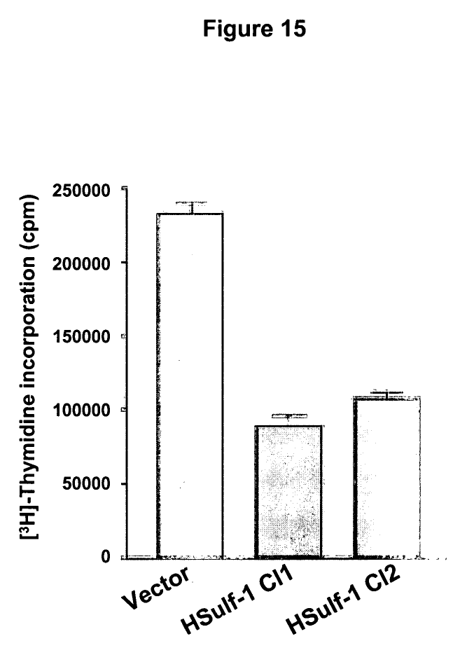

To determine whether this modulation might result in decreased cell proliferation and DNA synthesis, these properties were assessed using the MTT assay and [³H]-thymidine incorporation, respectively. 012SCC-HSulf-1 clones 1 and 2 proliferated more slowly compared to vector transfected control. This decrease in cell number of 012SCC HSulf-1 expressing clonal lines was correlated with decreased DNA synthesis (FIG. 15).

### Example 39

### HSulf-1 Inhibits HGF Mediated Motility and Invasion

To determine whether the inhibition of Akt activation is reflected in a change in motility or invasion of SCCHN cells, the motility of vector-transfected 012SCC and HSulf-1-transfected 012SCC clones 1 and 2 was evaluated with and without HGF treatment. Motility induced by 40 ng/ml HGF was significantly decreased in both HSulf-1 expressing 012SCC clones compared to the vector transfected control. Similar results were obtained when Matrigel invasion assays were performed. The HSulf-1 012SCC cells did not exhibit invasion of the basement membrane even in the presence of HGF, as compared to vector transfected controls.

### Example 40

### HSulf-1 Modulates Staurosporine and Cisplatin Mediated Apoptosis

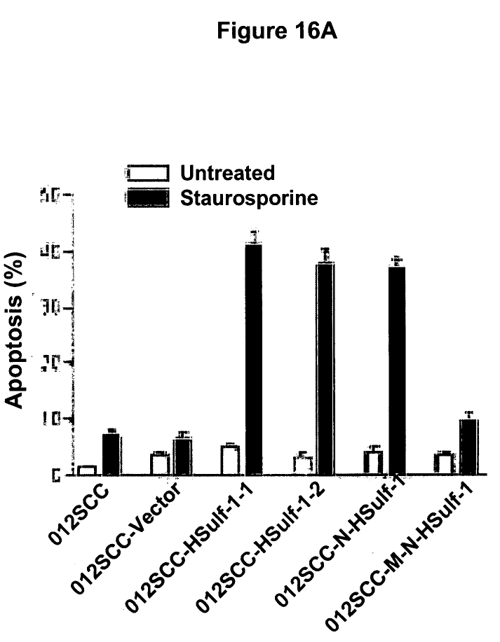

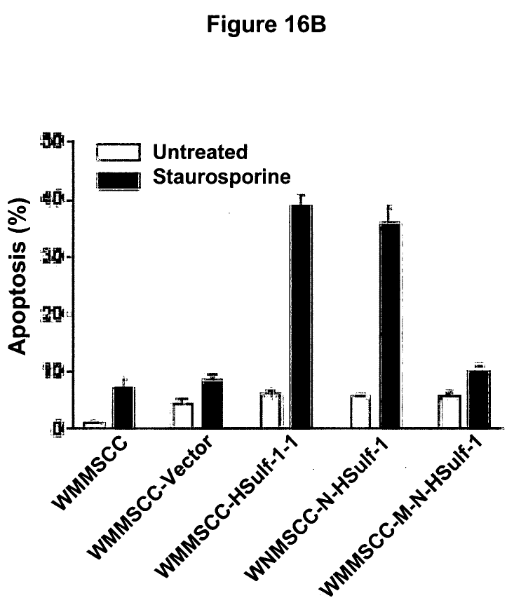

To examine the effects of HSulf-1 re-expression on apoptosis, stable transfectants were treated for 5 hours with 1 μM staurosporine, a broad spectrum kinase inhibitor that induces apoptosis in a wide variety of cells. Cells were then stained with DAPI and examined for apoptotic morphological changes (nuclear fragmentation) by fluorescence microscopy. These analyses indicated that staurosporine induced little apoptosis in parental or vector-transfected cells. In contrast, staurosporine induced apoptosis in 40% of HSulf-1-transfected cells (FIG. 16A). Similar results were observed in WMMCC cells transiently transfected with FL-HSulf-1, compared to parental or vector transfected controls. To confirm that this modulation of apoptosis reflected the sulfatase activity of HSulf-1, 012SCC and WMMSCC cells were transiently transfected with a C terminal truncation construct (N-Sulf) and active site mutant (Mut-N-Sulf). Transfection with the N-terminal fragment containing the entire sulfatase domain (N-Sulf) enhanced the ability of staurosporine to induce apoptosis. Importantly, site-directed mutagenesis of the putative catalytic cysteines C⁸⁷ and C⁸⁸ in N-Sulf-1 (Mut-N-Sulf-1) abolished the ability of HSulf-1 to modulate apoptosis (FIG. 16A), indicating that sulfatase activity is required for this modulation. HSulf-1 by itself did not induce apoptosis, but instead modulated the sensitivity of cells to other stimuli. These results were confirmed by flow cytometry with 7AAD staining in vector and HSulf-1-012SCC (FIG. 16B) and transiently transfected WMMSCC cells as compared to 012SCC and WMMSCC parental or vector transfected cells.

To determine whether HSulf-1 modulates apoptosis induced by cisplatin, the most commonly used drug to treat SCCHN patients, 012SCC HSulf-1 clones 1 and 2 were treated with 5 μM cisplatin for 24 hours and the percent of apoptotic cells was determined as described in Example 35. HSulf-1 modulated the apoptosis induced by cisplatin and the extent of apoptosis correlated with the levels of HSulf-1 expression.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-2. (canceled)

3. A method for killing a tumor cell, said method comprising administering to said tumor cell a nucleic acid that encodes an HSulf-1 polypeptide.

4. The method of claim 3, wherein said HSulf-1 polypeptide has the amino acid sequence set forth in SEQ ID NO:1 or a fragment thereof.

5. The method of claim 3, wherein the amino acid sequence of said HSulf-1 polypeptide comprises a variant relative to the amino acid sequence set forth in SEQ ID NO:1.

6. The method of claim 3, wherein a vector comprising said nucleic acid is administered to said tumor cell.

7. A method for killing a tumor cell, said method comprising administering to said tumor cell a purified HSulf-1 polypeptide.

8. The method of claim 7, wherein said HSulf-1 polypeptide has the amino acid sequence set forth in SEQ ID NO:1 or a fragment thereof.

9. The method of claim 7, wherein the amino acid sequence of said HSulf-1 polypeptide comprises a variant relative to the amino acid sequence set forth in SEQ ID NO:1

10-12. (canceled)

13. A method for determining whether a tumor will respond to treatment with a chemotherapeutic agent, said method comprising determining the level of HSulf-1 mRNA or polypeptide in said tumor.

14. The method of claim 13, wherein said chemotherapeutic agent is staurosporine, cisplatin, gemcitabine, topotecan, doxorubicin, or taxol.

15. The method of claim 13, wherein said HSulf-1 mRNA level is measured by reverse transcriptase PCR or light cycler PCR.

16. The method of claim 13, wherein said HSulf-1 polypeptide level is measured by antibody screening.

17. The method of claim 13, wherein said tumor is an ovarian tumor, a liver tumor, a squamous cell tumor, a breast tumor, or a pancreatic tumor.

18-20. (canceled)
